(19) United States
(12) Patent Application Publication
(10) Pub. No.: US 2017/0114029 A1
(43) Pub. Date: Apr. 27, 2017

(54) TREATMENT OF CHAGAS DISEASE

(71) Applicants: UNIVERSITY OF DUNDEE, Dundee (GB); MEDIVIR AB, Huddinge (SE); CONSEJO SUPERIOR DE INVESTIGACIONES CIENTIFICAS, Madrid (ES); SWISS TROPICAL AND PUBLIC HEALTH INSTITUTE, Basel (CH); SYNGENE INTERNATIONAL LIMITED PLC, Bangalore (IN)

(72) Inventors: Pia KAHNBERG, Stockholm (SE); Nils-Gunnar JOHANSSON, Flen (SE); Shahienaz HAMPTON, Dundee (GB); Justin HARRISON, Dundee (GB); Sandipan SARKAR, Bangalore (IN); Dolores GONZALES, Madrid (ES); Ian GILBERT, Dundee (GB)

(21) Appl. No.: 15/317,638

(22) PCT Filed: Jun. 9, 2015
(86) PCT No.: PCT/GB2015/051683

§ 371 Date: Dec. 9, 2016

(30) Foreign Application Priority Data
Jun. 11, 2014 (GB) 1410407.9

(51) Int. Cl. C07D 239/96; C07D 405/04; C07D 471/04; C07D 413/04; C07D 401/04

(52) U.S. Cl. CPC C07D 239/96; C07D 413/04; C07D 401/04; C07D 471/04; C07D 405/04

(57) ABSTRACT

The invention provides compounds of the formula: wherein L¹ and L² are independently selected from O and S; R¹ is C₃-C₆ straight or branched alkyl, C₃-C₇cycloalkyl, C₅-C₇cycloalkenyl, adamantly, phenyl or saturated heterocyclyl, any of which being optionally substituted; R² is H, methyl or ethyl; R⁵ is NRxCORy, NRxRy, CH₂COCH₃, CH₂C≡N, or a 5- or 6-membered heteroaryl group which is optionally substituted; X, Y and Z are independently N or CH; Rx is independently H or C₁-C₄alkyl; Ry is independently H, CrC4alkyl, phenyl or benzyl, either of which is optionally substituted; n is 0-3; salts, hydrates and N-oxides, wherein the optional substituents are further defined in the claims. The compounds have utility in the prophylaxis or treatment of trypanosomal diseases, such as T. cruzi (Chagas disease).

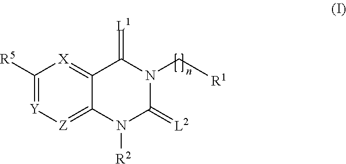

### FIELD OF THE INVENTION

This invention relates to quinazoline 2-4-diones and related aza analogues which have utility in the treatment of tyrpanosomal diseases, such as Trypanosoma cruzi (Chagas disease).

### BACKGROUND TO THE INVENTION

Trypanosoma cruzi (T. cruzi) is an obligate intracellular protozoan parasite. In mammalian hosts T. cruzi cycles between a trypomastigote stage which circulates in the blood and the amastigote stage which replicates in the cytoplasm of infected host cells (primarily muscle).

T. cruzi is the etiological agent of Chagas disease and is ranked as the most serious parasitic disease in the Americas, with an economic impact far outranking the combined effects of other parasitic diseases such as malaria, schistosomiasis, and leishmania. Chagas Disease affects up to 20 million individuals primarily in the Americas where the insect vectors are present and where zoonotic transmission cycles guarantee a steady source of parasites. T. cruzi infection has its greatest human impact in areas of Latin America where housing conditions bring people, infected animals, and vector insects into close proximity. More than 90 million are at risk of infection in endemic areas, and roughly 50,000 children and adults die of chronic Chagas disease every year due to lack of effective treatments. Additionally, 2-5% of fetus carried by infected mothers in endemic areas are either aborted or born with congenital Chagas disease. Loss of revenue in terms of productivity lost due to sickness and medical costs have an overwhelming effect on economic growth of these countries.

Recently, increasing travel and immigration have brought T. cruzi infection into the spotlight globally, even in areas where transmission has previously been absent or very low. T. cruzi has spread beyond the borders of Latin America and has been detected in Europe, Asia, and the United States. In the U.S., 50-100 thousand serologically positive persons progressing to the chronic phase of Chagas disease are present, and the number of infected immigrants in developed countries is increasing. It is expected that, due to the exponential increase in emigration from Latin America, Chagas disease may become a serious health issue in North America and Europe in the next decade.

Congenital and transfusion/transplantation-related transmissions are thus becoming increasingly recognized as significant threats. As the number of infected individuals grows, transmission of T. cruzi to non-infected individuals through blood transfusion and organ transplants from the infected immigrant donors is emerging as a route for T. cruzi transmission in more developed nations.

Each year, 15 million units of blood are transfused and approximately 23,000 organ transplants are performed in the United States alone, and presently almost none of the blood supply is tested for T. cruzi. A few cases of infection by T. cruzi through organ donation have already been reported to United States Centers for Disease Control since 2001. It has therefore become apparent that the screening of blood and organ donors is necessary not only in Latin America but also in developed countries that receive immigrants from endemic areas.

Diagnosis of T. cruzi infection is challenging for a number of reasons. The initial infection is seldom detected except in cases where infective doses are high and acute symptoms very severe, as in localized outbreaks resulting from oral transmissions. Classical signs of inflammation at proposed sites of parasite entry (e.g. “Romaña's sign”) or clinical symptoms other than fever, are infrequently reported. As a result, diagnosis is very rarely sought early in the infection, when direct detection of parasites may be possible. In the vast majority of human cases T. cruzi infection evolves undiagnosed into a well-controlled chronic infection wherein circulating parasites or their products are difficult to detect even with the use of amplification techniques. A “conclusive” diagnosis of T. cruzi infection is often reached only after multiple serological tests and in combination with epidemiological data and (occasionally) clinical symptoms. Further complicating matters, some researchers have reported positive PCR and clinical disease in patients with negative serology. A corollary of the difficult diagnosis is that putative pharmaceutical agents should preferably have very good safety profiles, as they may be administered to patients with unconfirmed pathology. Other challenges with Chagas treatment include the endemic poverty in many of the areas in which it is found, which rule out the use of sophisticated biologicals and other preparations with stringent refrigeration needs or intravenous dosing regimes.

Clark et al Biorg Med Chem 20 (2012) 6019-6033 has described a family of quinazoline-2-4-diones with modest activities against the parasite Trypansomosa brucei which causes HAT or sleeping sickness in Africa. Characteristic for these compounds is that activity against T. brucei is enhanced with larger substituents at the N-1 position. The only substituents at the 5 position are hydrogen or chloro.

### BRIEF DESCRIPTION OF THE INVENTION

In accordance with a first aspect of the invention, there is provided a compound of formula I:

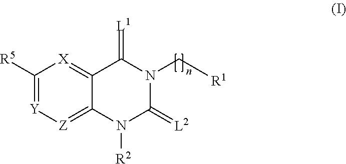

wherein
L¹ and L² are independently selected from O and S;
R¹ is C₃-C₆ straight or branched alkyl, C₃-C₇cycloalkyl, C₅-C₇cycloalkenyl, adamantyl, phenyl or saturated heterocyclyl, any of which being optionally substituted with 1-3 substituents selected from halo, C₁-C₆alkyl, C₃-C₆cycloalkyl, C₁-C₆haloalkyl, C₁-C₆alkoxy, ORy, SRy, N₃, NRxRy, CORy, COORy, and CONRxRy;
R² is H, methyl or ethyl;
R⁵ is NRxCORy, NRxRy, CH₂COCH₃, CH₂C≡N, or a 5- or 6-membered heteroaryl group which is optionally substituted with 1-3 substituents independently selected from halo, C₁-C₆ alkyl, C₃-C₆ cycloalkyl, C₁-C₆haloalkyl, C₁-C₆alkoxy, ORy, SRy, N₃, NRxRy, CORy, COORy, and CONRxRy;
X, Y and Z are independently N or CH;
Rx is independently H or C₁-C₄alkyl;
Ry is independently H, C₁-C₄alkyl or phenyl or benzyl, either of which is optionally substituted with 1-3 substituents selected from halo, C₁-C₆alkyl, C₃-C₆cycloalkyl, C₁-C₆haloalkyl, C₁-C₆alkoxy, COC₁-C₆alkyl;
n is 0-3
or a pharmaceutically acceptable salt, hydrate or N-oxide thereof.

In one embodiment of the invention, X, Y and Z are each CH.

In an alternative embodiment, one of X, Y and Z is N, and the others are each CH, typically according to this embodiment, Z is N, and X and Y are each CH.

In one embodiment of the invention one of L₁ and L₂ is O, and the other S.

A currently favoured embodiment of the invention has the formula

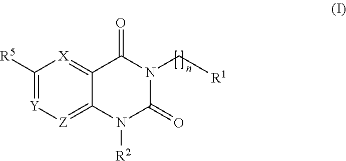

where X, Y, Z, R¹, R², R⁵ and n are as defined for formula I.

In one embodiment of the invention, n is 2.

In an alternative embodiment of the invention, n is 0.

In one embodiment of the invention, R¹ is optionally substituted C₃-C₇cycloalkyl. In an alternative embodiment, R¹ is optionally substituted C₅-C₇cycloalkenyl.

In compounds wherein R¹ is C₅-C₇cycloalkenyl, the double bond is typically located in the 1-position of the cycloalkenyl moiety.

In compounds wherein R¹ is substituted cyclohexyl, the substituent is typically located in the 4-position of the cyclohexyl moiety.

Typical substituents to the cycloalkyl or cycloalkenyl moiety include C₁-C₄alkyl, such as ethyl, isopropyl and tert.butyl.

In certain embodiments the optional substituents to the cycloalkyl or cycloalkenyl moiety include halo such as fluoro or chloro, preferably fluoro, and C₁-C₆haloalkyl such as fluoromethyl, difluoromethyl and trifluoromethyl.

In certain other embodiments, the optional substituent to the cycloalkyl or cycloalkenyl moiety is C₃-C₆ alkyl, such as cyclopropyl or cyclobutyl.

Alternative substituents to the cycloalkyl or cycloalkenyl moiety include C₁-C₄alkoxy, such as methoxy and ethoxy.

In a typical embodiment, n is 2 and R¹ is cyclohexyl or cyclohexenyl, either of which is optionally substituted.

In a further typical embodiment, n is 0 and R¹ is cyclopentyl or cyclohexyl either of which is optionally substituted.

A typical configuration of R¹ is cyclohexyl which is substituted in the 4-position. Typically, the substituent is C₁-C₄alkyl, such as isopropyl or a C₃-C₆ cycloalkyl, such as cyclopropyl.

In a typical embodiment of the invention, n is 0 and R¹ is cyclohexyl which is substituted in the 4-position with C₁-C₄alkyl, such as isopropyl.

R² is typically H.

In one embodiment of the invention, R⁵ is NRxCORy. Typically according to this embodiment, Rx is H and Ry is C₁-C₄alkyl. Preferably Rx is H and Ry is CH₃.

In an alternative embodiment of the invention, R⁵ is NRxRy. Typically according to this embodiment, Rx is H and Ry is C₁-C₄alkyl such as CH₃. In a specific embodiment Rx and Ry are both H In an alternative embodiment of the invention, R⁵ is a 5- or 6-membered heteroaryl group which is optionally substituted. In one embodiment, R⁵ is pyridyl.

In preferred embodiments of the invention, R⁵ is NH₂ or NHCOCH₃.

A further aspect of the invention provides a method for the prophylaxis or treatment of trypanosomal infection comprising the administration of a compound of formula I to a subject suffering from or likely to be exposed to said trypanosomal infection. A related aspect of the invention provides the use of a compound of formula I in the treatment or prophylaxis of trypanosomal infection. A further related aspect provides the use of the compounds of formula I in the manufacture of a medicament for the treatment or prophylaxis of trypanosomal infection.

A further aspect of the invention provides a method for the treatment of trypanosomal infection comprising the administration of a compound of formula I to a subject suffering from or likely to be exposed to said trypanosomal infection. A related aspect of the invention provides the use of a compound of formula I in the treatment of trypanosomal infection. A further related aspect provides the use of the compounds of formula I in the manufacture of a medicament for the treatment of trypanosomal infection. Other related aspects provide, a compound of formula I for use in the treatment of trypanosomal infection, and a compound of formula I for use in the treatment of a T cruzi infection.

In some embodiments of the invention, the trypanosomal infection is a T cruzi infection. Typically the method or use of the invention relates to treatment of an ongoing infection in human subjects.

The agents according to the present invention are believed to be suitable for those diseases in which the pathogen is present in organs such as the liver, spleen or kidney, and in particular to muscles such as heart.

In a further aspect, the invention provides a compound of formula I for use as a medicament.

In another aspect, the invention provides a pharmaceutical composition comprising one or more compounds of any of the formulae herein and a pharmaceutically acceptable carrier, vehicle or diluent therefor.

In another aspect, the invention provides a kit comprising an effective amount of one or more compounds of the formulae herein in unit dosage form, together with instructions for administering the compound to a subject suffering from or susceptible to a trypanosomal infections, such as Chagas disease.

As used herein, the following terms have the meanings as defined below, unless otherwise noted:

“Cₘ-Cₙalkyl” on its own or in composite expressions such as Cₘ-Cₙhaloalkyl, etc. represents a straight or branched alkyl radical having the number of carbon atoms designated, e.g. C₁-C₄alkyl means an alkyl radical having from 1 to 4 carbon atoms. C₁-C₆alkyl has a corresponding meaning, including also all straight and branched chain isomers of pentyl and hexyl. Preferred alkyl radicals for use in the present invention are C₁-C₆alkyl, including methyl, ethyl, n-propyl, isopropyl, n-butyl, isobutyl, sec.butyl, tert.butyl, n-pentyl and n-hexyl, especially C₁-C₄alkyl such as methyl, ethyl, n-propyl, isopropyl, tert.butyl, n-butyl and isobutyl.

The term “Cₘ-Cₙhaloalkyl” as used herein represents Cₘ-Cₙalkyl wherein at least one C atom is substituted with a halogen (e.g. the Cₘ-Cₙhaloalkyl group may contain one to three halogen atoms), preferably chloro or fluoro. Typical haloalkyl groups are C₁-C₂haloalkyl, in which halo suitably represents fluoro. Exemplary haloalkyl groups include fluoromethyl, difluoromethyl and trifluoromethyl.

The term “Me” means methyl, and “MeO” means methoxy.

“Cₘ-Cₙalkoxy” represents a radical Cₘ-Cₙalkyl-O— wherein Cₘ-Cₙalkyl is as defined above. Of particular interest is C₁-C₄alkoxy which includes methoxy, ethoxy, n-propoxy, isopropoxy, t-butoxy, n-butoxy and isobutoxy. Methoxy and isopropoxy are typically preferred. C₁-C₆alkoxy has a corresponding meaning, expanded to include all straight and branched chain isomers of pentoxy and hexoxy.

The term “amino” represents the radical —NH₂.

The term “halo” represents a halogen radical such as fluoro, chloro, bromo or iodo. Typically, halo groups are fluoro or chloro.

The term “heterocyclyl” represents a stable saturated monocyclic 3-7 membered ring containing 1 or 2 heteroatoms independently selected from O and N. A typical configuration of heterocyclyl is a 5-7 membered ring containing 1 heteroatom selected from O and N. A further typical configuration of heterocyclyl is a 5-7 membered ring containing 2 heteroatoms selected from O and N. Preferred heterocyclyl is a 5 or 6 membered ring.

The term “heteroaryl” represents a stable monocyclic aromatic ring containing 1-4 heteroatoms independently selected from O, S and N, having 5 or 6 ring atoms. In one embodiment of the invention the stable monocyclic ring contains one heteroatom selected from O, S and N has 5 or 6 ring atoms. In a second embodiment of the invention the stable monocyclic aromatic ring contains two heteroatoms independently selected from O, S and N, and has 5 or 6 ring atoms. In a third embodiment the stable monocyclic aromatic ring contains three heteroatoms independently selected from O, S and N, has 5 or 6 ring atoms. In a fourth embodiment the stable monocyclic aromatic ring system contains four heteroatoms independently selected from O, S and N, and has 5 or 6 ring atoms. The heteroaryl is optionally substituted with one, two or three substituents independently selected from halo, C₁-C₆alkyl, C₃-C₆cycloalkyl, C₁-C₆haloalkyl, C₁-C₆alkoxy, ORy, SRy, N₃, NRxRy, CORy, COORy, and CONRxRy; wherein R is independently H or C₁-C₄alkyl and Ry is independently H, C₁-C₄alkyl, phenyl or benzyl.

The term “C₃-Cₙcycloalkyl” represents a cyclic monovalent alkyl radical having the number of carbon atoms indicated, e.g. C₃-C₇cycloalkyl means a cyclic monovalent alkyl radical having from 3 to 7 carbon atoms. Preferred cycloalkyl radicals for use in the present invention are C₃-C₄alkyl i.e. cyclopropyl and cyclobutyl.

The term “C₅-Cₙcycloalkenyl” represents a cyclic monounsaturated monovalent alkyl radical having the number of carbon atoms indicated, e.g. C₅-C₇cycloalkenyl means a cyclic monounsaturated monovalent alkyl radical having from 5 to 7 carbon atoms. Preferred cycloalkenyl radicals for use in the present invention are C₅-C₆alkyl i.e. cyclopentenyl and cyclohexenyl. Unless specifically indicated, the double bond in the cycloalkenyl moiety can be located anywhere in the ring. For example, 1-cyclohexenyl means a cyclohexenyl radical wherein the double bond is located at the carbon of attachment, i.e.

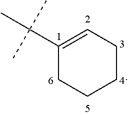

As used herein, the term “═O” forms a carbonyl moiety when attached to a carbon atom. It should be noted that an atom can only carry an oxo group when the valency of that atom so permits.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

The term “therapeutically effective amount” means an amount effective to treat, cure or ameliorate a disease, illness or sickness.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange.

Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulphonate.

The compounds of the invention can be administered as pharmaceutically acceptable prodrugs which release the compounds of the invention in vivo. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in “Design and Application of Prodrugs, Textbook of Drug Design and Development, Chapter 5, 113-191 (1991); Bundgaard, et al., Journal of Drug Deliver Reviews, 8:1-38(1992); and Bernard Testa and Joachim Mayer, “Hydrolysis In Drug and Prodrug Metabolism—Chemistry, Biochemistry and Enzymology,” John Wiley and Sons, Ltd. (2003).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Related terms, are to be interpreted accordingly in line with the definitions provided above and the common usage in the technical field.

The present invention also includes isotope-labelled compounds of formula I or any subgroup of formula I, wherein one or more of the atoms is replaced by an isotope of that atom, i.e. an atom having the same atomic number as, but an atomic mass different from, the one(s) typically found in nature. Examples of isotopes that may be incorporated into the compounds of formula I or any subgroup of formula I, include but are not limited to isotopes of hydrogen, such as ²H and ³H (also denoted D for deuterium and T for tritium, respectively), carbon, such as ¹¹C, ¹³C and ¹⁴C, nitrogen, such as ¹³N and ¹⁵N, oxygen, such as ¹⁵O, ¹⁷O and ¹⁸O, phosphorus, such as ³¹P and ³²P, sulphur, such as ³⁵S, fluorine, such as ¹⁸F, chlorine, such as ³⁶Cl, bromine such as ⁷⁵Br, ⁷⁶Br, ⁷⁷Br and ⁸²Br, and iodine, such as ¹²³I, ¹²⁴I, ¹²⁵I and ¹³¹I. The choice of isotope included in an isotope-labelled compound will depend on the specific application of that compound. For example, for drug or substrate tissue distribution assays, compounds wherein a radioactive isotope such as ³H or ¹⁴C is incorporated will generally be most useful. For radio-imaging applications, for example positron emission tomography (PET) a positron emitting isotope such as ¹¹C, ¹⁸F, ¹³N or ¹⁵O will be useful. The incorporation of a heavier isotope, such as deuterium, i.e. ²H, may provide greater metabolic stability to a compound of formula I or any subgroup of formula I, which may result in, for example, an increased in vivo half-life of the compound or reduced dosage requirements.

Isotope-labelled compounds of formula I or any subgroup of formula I can be prepared by processes analogous to those described in the Schemes and/or Examples herein below by using the appropriate isotope-labelled reagent or starting material instead of the corresponding non-isotope-labelled reagent or starting material, or by conventional techniques known to those skilled in the art.

The N-oxides of compounds of the invention can be prepared by methods known to those of ordinary skill in the art. For example, N-oxides can be prepared by treating an unoxidized form of the compound of the invention with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of the invention can be prepared from the N-oxide of an appropriate starting material.

Examples of N-oxides of the invention include those with the partial structures:

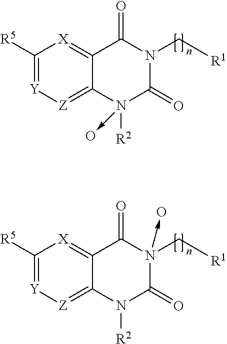

Compounds of the invention in unoxidized form can be prepared from N-oxides of the corresponding compounds of the invention by treating with a reducing agent (e.g. sulphur, sulphur dioxide, triphenyl phosphine, lithium borohydride, sodium borohydride, phosphorus dichloride, tribromide, or the like) in an suitable inert organic solvent (e.g. acetonitrile, ethanol, aqueous dioxane or the like) at 0 to 80° C.

In some cases, the compounds of formula I are represented as a defined stereoisomer. The absolute configuration of such compounds can be determined using art-known methods such as, for example, X-ray diffraction or NMR and/or implication from start materials of known stereochemistry. Pharmaceutical compositions in accordance with the invention will preferably comprise substantially stereoisomerically pure preparations of the indicated stereoisomer.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound is synthesized by stereospecific methods of preparation. These methods will advantageously employ enantiomerically pure starting materials.

The diastereomeric racemates of the compounds of formula I can be obtained separately by conventional methods. Appropriate physical separation methods that may advantageously be employed are, for example, selective crystallization and chromatography, e.g. column chromatography.

### Pharmaceutical Compositions

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type.

The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. Solid compositions of a similar type may also be employed as fillers in soft and hard-filled gelatine capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, disorders are treated or prevented in a subject, such as a human or other animal, by administering to the subject a therapeutically effective amount of a compound of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

The dosage for the instant compounds can vary according to many factors, including the type of disease, the age and general condition of the patient, the particular compound administered, and the presence or level of toxicity or adverse effects experienced with the drug. A representative example of a suitable dosage range is from as low as about 0.025 mg to about 1000 mg. However, the dosage administered is generally left to the discretion of the physician.

A wide variety of pharmaceutical dosage forms for mammalian patients can be employed. If a solid dosage is used for oral administration, the preparation can be in the form of a tablet, hard gelatin capsule, troche or lozenge. The amount of solid carrier will vary widely, but generally the amount of the present compound will be from about 0.025 mg to about 1 g, with the amount of solid carrier making up the difference to the desired tablet, hard gelatin capsule, troche or lozenge size. Thus, the tablet, hard gelatin capsule, troche or lozenge conveniently would have, for example, 0.025 mg, 0.05 mg, 0.1 mg, 0.5 mg, 1 mg, 5 mg, 10 mg, 25 mg, 100 mg, 250 mg, 500 mg, or 1000 mg of the present compound. The tablet, hard gelatin capsule, troche or lozenge is given conveniently once, twice or three times daily.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors.

In certain embodiments, a therapeutic amount or dose of the compounds of the present invention may range from about 0.1 mg/Kg to about 500 mg/Kg, alternatively from about 1 to about 50 mg/Kg. In general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents. Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms. It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The invention also provides for pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration. The terms “co-administration” or “combined administration” or the like as utilized herein are meant to encompass administration of the selected therapeutic agents to a single patient, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the invention and a co-agent, are both administered to a patient simultaneously in the form of a single entity or dosage. The term “non-fixed combination” means that the active ingredients, e.g. a compound of the invention and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulphate and magnesium stearate, as well as colouring agents, releasing agents, coating agents, sweetening, flavouring and perfuming agents, preservatives and antioxidants can also be present in the composition, according to the judgment of the formulator.

In addition to the definitions above, the following abbreviations are used in the examples and synthetic schemes below. If an abbreviation is not defined, it has its generally accepted meaning.

### ACN Acetonitrile

### DCM Dichloromethane

CDI Carbonyl diimidazole
BOP (Benzotriazol-1-yloxy)tris(dimethylamino)phosphonium hexafluorophosphate

### DIEA Diisopropylethylamine

### DMA N,N-dimethylacetamide

### DMAP 4-Dimethylaminopyridine

### DMF N,N-Dimethylformamide

EDC 1-Ethyl-3-(3-dimethylaminopropyl)carbodiimide
EtOAc Ethyl acetate

### Et₃N Triethylamine

### EtOH Ethanol

Et₂O Diethyl ether
LC Liquid chromatography
HATU O-(7-Azabenzotriazol-1-yl)-N,N,N′,N′-tetramethyluronium hexafluorophosphate
HOAc Acetic acid
HPLC High performance liquid chromatography

### MeOH Methanol

### IPA Isopropylalcohol

### NMM N-Methylmorpholine

### Ph Phenyl

pyBOP Benzotriazol-1-yl-oxytripyrrolidinophosphonium hexafluorophosphate
TBAF Tetrabutylammonium fluoride
TBDMSi Tert.butyldimethylsilyl

### THF Tetrahydrofuran

TFA Trifluoroacetic acid

### General Synthetic Methods

Compounds of the present invention may be prepared by a variety of methods e.g. as depicted in the illustrative synthetic schemes shown and described below. The starting materials and reagents used are available from commercial suppliers or can be prepared according to literature procedures set forth in references, using methods well known to those skilled in the art.

Compound of the invention wherein X, Y and Z are all CH, and R⁵ is NHCOMe or NH₂, can be prepared from commercially available 5-acetamido-2-aminobenzoic acid. A method wherein a cyclic anhydride is formed in a first step is illustrated in Scheme 1.

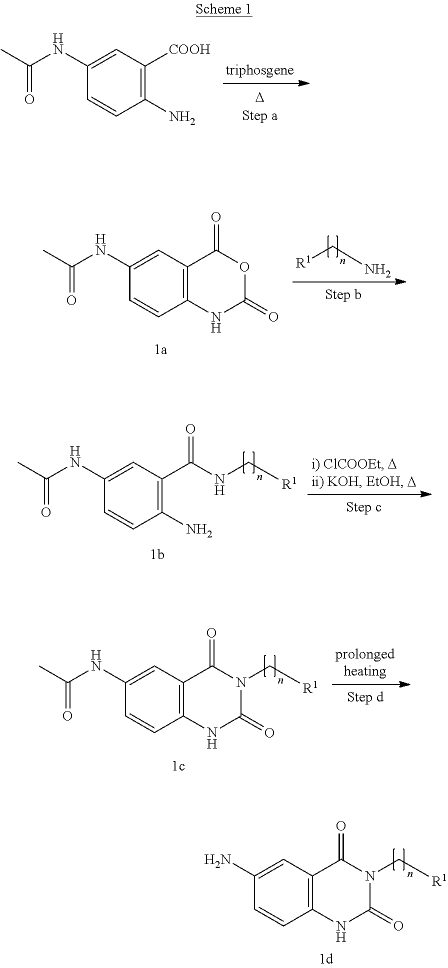

Treatment of 5-acetamido-2-aminobenzoic acid with triphosgene or the like under elevated temperature provides the cyclic anhydride 1a. The afforded anhydride is then opened with a desired amine R¹—(CH₂)ₙ—NH₂ in the presence of DMAP or similar, which provides the amide 1b. Carbonylation and ring formation effected for instance by reaction with ethylchloroformate or the like, followed by treatment with hydroxide or equivalent then yields the bicyclic compound 1c. The carbonylation-ring closing step may alternatively be effected by any other suitable reagent such as CDI. Prolonged heating in the presence of KOH of the afforded compound removes the acetyl moiety from the nitrogen, thus giving the corresponding amine, i.e. compounds of the invention wherein R⁵ is NH₂.

Compounds of the invention wherein X, Y and Z are all CH, and R⁵ is NH₂, may alternatively be prepared from commercially available 5-aminoisatoic anhydride, as illustrated in Scheme 2.

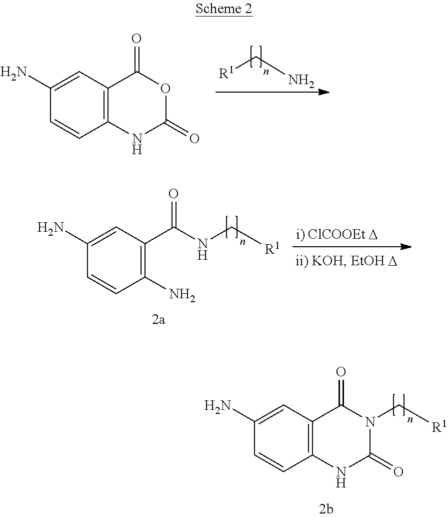

Opening of the anhydride with a desired amine R¹—(CH₂)ₙ—NH₂, followed by carbonylation-ring closure as described above, provides the aniline compound 2b.

Compounds of the invention wherein X, Y and Z are all CH, and R⁵ is an optionally substituted 5- or 6-membered heteroaryl group can be prepared as illustrated in Scheme 3.

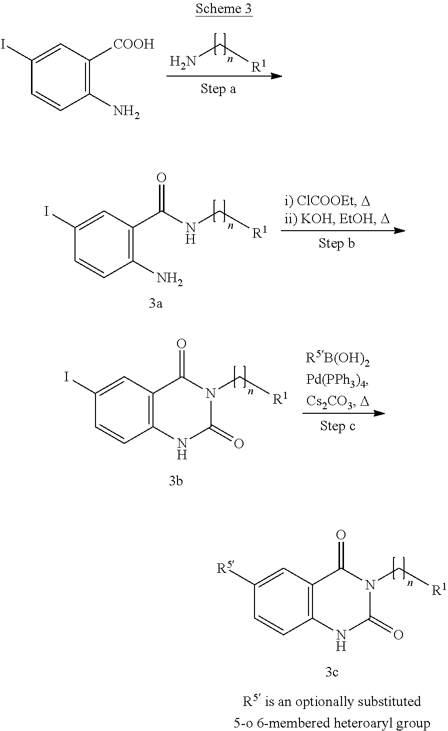

Reaction of commercially available 2-amino-5-iodobenzoic acid with the suitable amine, R¹—(CH₂)ₙ—NH₂, under peptide coupling conditions, i.e. using a peptide coupling agent like HATU, pyBOP, EDC or the like in the presence of a base like NMM, DIEA or similar, in a solvent such as DMF, provides the amide 3a. Carbonylation and ring formation can then be effected by reaction with for instance ethylchloroformate or the like, followed by treatment with hydroxide or equivalent. Other carbonylation-ring closing conditions may alternatively be used such as CDI. Introduction of the heteroaromatic ring is then effected using for instance a palladium catalysed cross coupling reaction, such as a Suzuki reaction or any suitable variant thereof, i.e. reaction with the desired organoboronic acid R^{5′}—B(OH)₂ in the presence of a Pd catalyst such as Pd(PPh₃)₄ and a suitable base such as K₂CO₃, Cs₂CO₃ or the like in a solvent like dioxane/H₂O at elevated temperature, thus providing the biarylic compound 3c.

Biarylic compounds of the invention X, Y and Z are all CH, may alternatively be obtained from the commercially available cyclic anhydride 6-bromo-1H-benzo[d][1,3]oxazine-2,4-dione, using similar procedures. A general overview is depicted in Scheme 4.

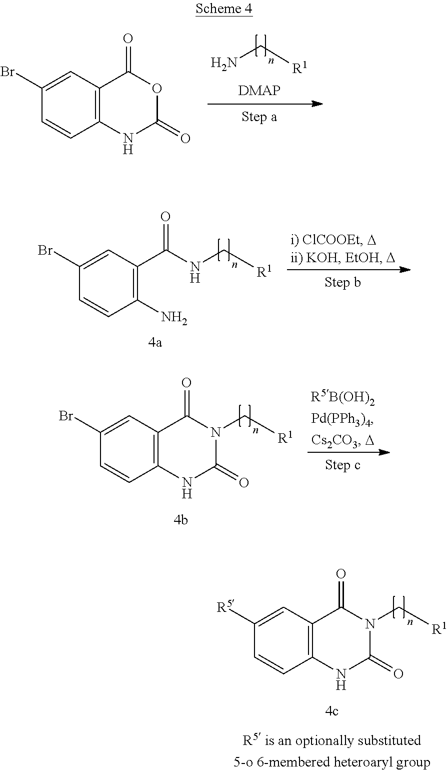

Opening of the anhydride with a desired amine R¹—(CH₂)ₙ—NH₂ in the presence of DMAP or similar, provides the amide (4a). Carbonylation and ring closure followed by biaryl coupling as described above then provides the target compound 4c.

Compounds of the invention wherein X and Y are CH, Z is N and R⁵ is NRxRy or NRxCORy can be prepared from a commercially available ester of methyl 2-aminonicotinic acid as depicted in Scheme 5.

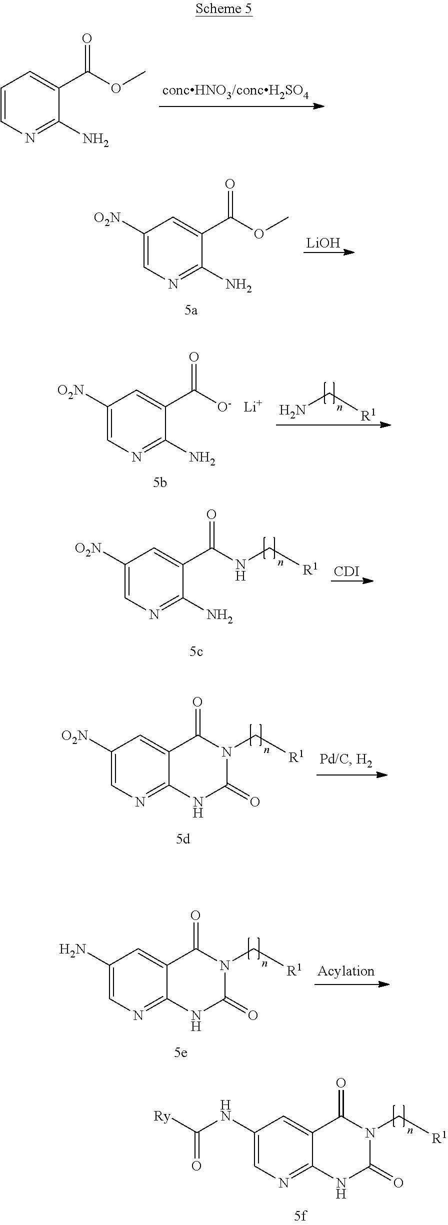

Nitration of methyl 2-aminonicotinic acid effected by treatment with a mixture of concentrated HNO₃ an H₂SO₄ followed by hydrolysis of the methyl ester by treatment with LiOH or similar provides the salt 5b. Subsequent coupling of a desired amine R¹—(CH₂)ₙ—NH₂ using standard peptide coupling conditions such as treatment with a coupling agent like pyBOP, EDC, HATU or the like in the presence of a base such as TEA or similar provides the amide 5c. Carbonylation and ring formation effected for instance by treatment with CDI, or conditions like ethylchloroformate or equivalent followed by KOH, provides the pyridopyrimidine derivative 5d. Reduction of the nitro function brought about by e.g. catalytic hydrogenation yields the amine 5e. If desired, the amino group can be acylated thus forming an amide, using the appropriate acylating conditions such as treatment with an anhydride in the presence of TEA, or treatment with a desired acyl halide such as the acyl chloride or the like. For example, treatment with acetic anhydride in the presence of TEA provides acetamide 5f. Alternatively, the amide may be formed by reaction with the desired acid using standard peptide coupling conditions.

An alternative approach to compounds of the invention wherein R⁵ is NRxCORy is illustrated in Scheme 6.

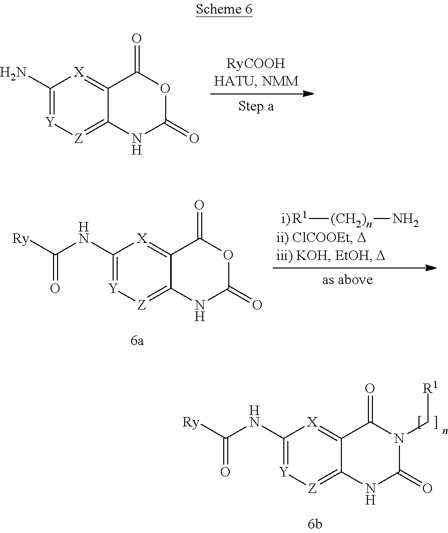

Coupling of the desired acid RyCOOH with the aniline using standard peptide coupling conditions provides amide 6a. Subsequent introduction of the amine R¹—(CH₂)ₙ—NH₂ followed by carbonylation and ring formation as described above, provides the final compound 6b.

Compounds of the invention wherein X, Y and Z are all CH, and R² is methyl or ethyl are obtained e.g. by alkylation of the isatoic anhydride as illustrated in Scheme 7.

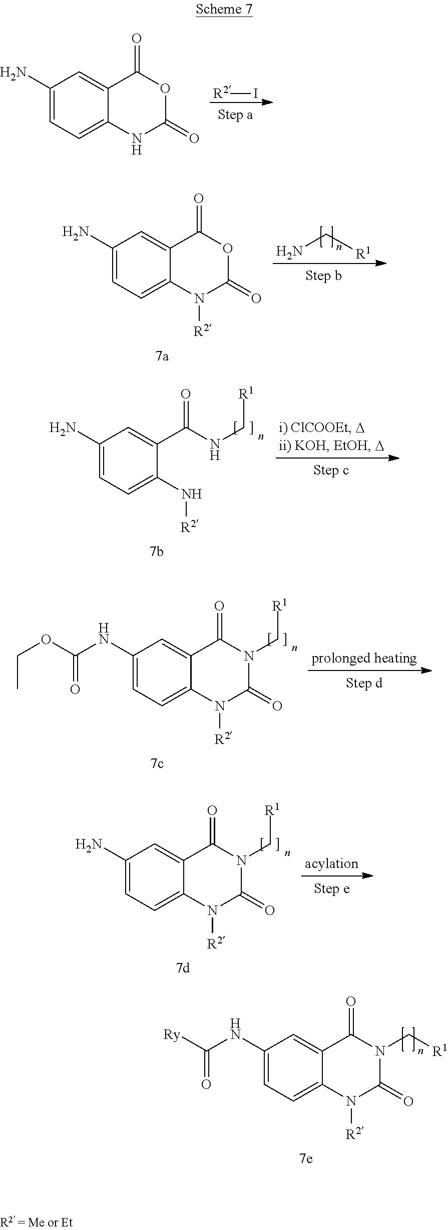

Selective alkylation of the ring-nitrogen of 5-aminoisatoic anhydride can be performed by reaction with methyl iodide or ethyl iodide or equivalent in a solvent like DMA or the like, thus providing the N-alkylated derivative 7a. Amide formation by reaction with a suitable R¹—(CH₂)ₙ—NH₂ followed by carbonylation and ring closure as described above, provides the intermediate carbamate 7c. Prolonged heating of the carbamate then provides amino derivative 7d. If desired, the afforded amine can subsequently be acylated as described above, thus affording acyl derivative 7e.

Compounds of the invention wherein R⁵ is NHRMe or NHREt, and R² is Me or Et can be prepared by N-alkylation of compound 1e, followed by N-deacetylation, as depicted in Scheme 8.

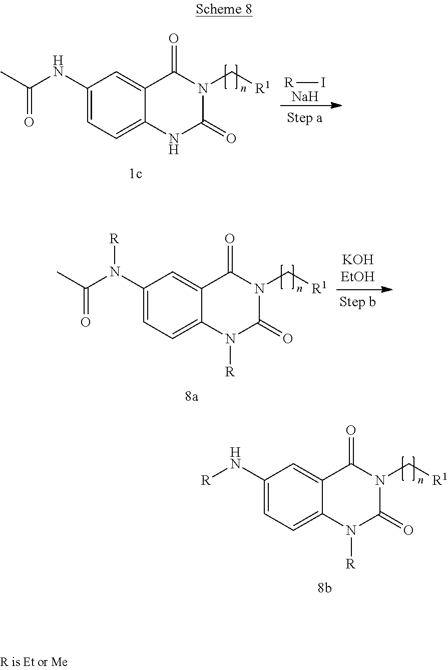

Amines, R¹—(CH₂)ₙNH₂, used in the above schemes are commercially available, or they can be prepared according to literature procedures or as described herein below. For example, alkyl substituted C₃-C₇cycloalkylamines can be prepared from the corresponding C₃-C₇cycloalkanone as illustrated in Scheme 9.

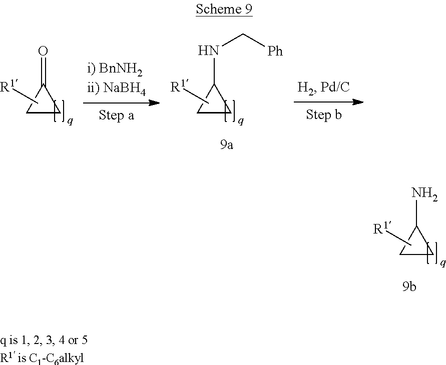

Subjection of the suitably substituted cycloalkanone to a reductive amination reaction, i.e. reaction with an amine such as benzylamine followed by reduction using a suitable reductive agent such as NaBH₄ or NaCNBH₃ or the like, provides benzylamine derivative 9a. Removal of the benzyl group effected for example by catalytic hydrogenation using a catalyst like palladium on carbon or the like, provides the desired alkyl substituted C₃-C₇cycloalkylamine 9b.

Compounds of the invention wherein L¹ is O, L² is S and R⁵ is NHC(═O)Ry or NH₂, can be prepared as outlined in Scheme 10.

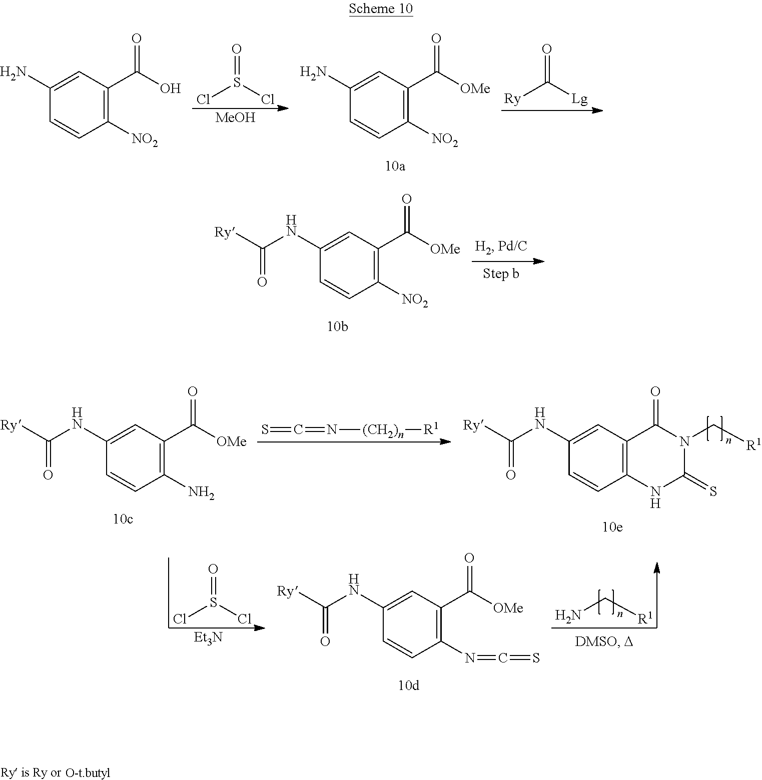

Esterification of commercially available 5-amino-2-nitrobenzoic acid using for instance conditions like thionyl chloride in methanol, or methanol in the presence of sulphuric acid, followed by N-acylation using conditions like treatment with an acylating agent Ry(C═O)Lg wherein Lg is a leaving group, such as acetyl chloride, benzoyl chloride or pivaloyl chloride in a solvent like DCM in the presence of DIPEA or the like, or by treatment with the appropriate anhydride in the presence of H₂SO₄ in a solvent like DCM, provides acyl derivative (10b). Reduction of the nitro group using standard conditions such as catalytic hydrogenation using a suitable catalyst, e.g. Pd/C, provides the corresponding amine (10c). The thioxoquinazolinone derivative (10e) can then be prepared either by reaction with a suitably substituted isothiocyanate, S═C═N—(CH₂)ₙR¹, optionally in the presence of a base like triethylamine or DMAP or similar in a solvent like toluene, acetonitrile, DMSO or the like, typically at an elevated temperature. Alternatively, the thioxoquinazolinone derivative (10e) can be prepared in a two-step reaction sequence; forming an intermediate isothiocyanate in a first step effected by reaction with thiophosgene in the presence of a base like triethylamine, sodium hydrogen carbonate or similar, followed by cyclization effected by reaction with an amine H₂N—(CH₂)ₙ—R¹ using a solvent like DMSO, DMF or similar, typically at an elevated temperature. In the case Ry′ is O-t.butyl, i.e. forming a Boc group together with the carbonyl to which it is attached, the whole Boc group can be removed by treatment with acid, such as treatment with TFA in DCM or equivalent, thus affording an amine, i.e. a compound of formula I wherein R⁵ is NH₂.

Compounds of the invention wherein R⁵ is an optionally substituted 5-6-membered heteroaryl group can be prepared using a similar strategy, starting from the appropriately substituted methyl anthranilate, as illustrated in Scheme 11.

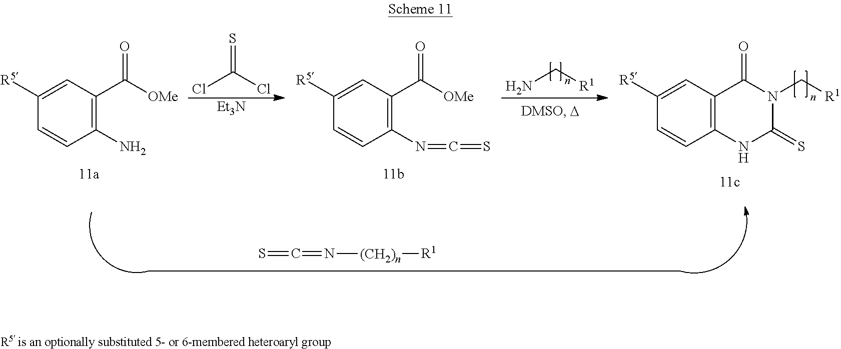

The thioxoquinazolinone derivative (11c) can then be prepared either by reaction of methyl anthranilate (11a) with a suitably substituted isothiocyanate, S═C═N—(CH₂)ₙ—R¹, optionally in the presence of a base like triethylamine or DMAP or similar in a solvent like toluene, acetonitrile, DMSO or the like, typically at an elevated temperature. Alternatively, the thioxoquinazolinone derivative (11c) can be prepared in a two-step reaction sequence; forming an intermediate isothiocyanate in a first step effected by reaction with thiophosgene in the presence of a base like triethylamine, sodiumhydrogen carbonate or similar, followed by cyclization effected by reaction with an amine H₂N—(CH₂)ₙ—R¹ using a solvent like DMSO, DMF or similar, typically at an elevated temperature.

Isothiocyanates S═C═N(CH₂)ₙR¹ used in the above schemes are commercially available, or they can be prepared from the desired primary amine H₂N(CH₂)ₙR¹ as outlined in Scheme 12.

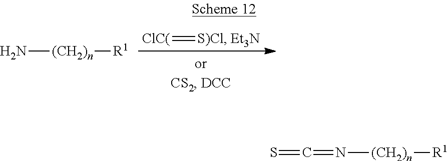

Treatment primary amine H₂N(CH₂)ₙR¹ with thiophosgene in the presence of a base like tertiary amine, e.g. triethylamine, in an aprotic solvent such as DCM or THF, provides the isothiocyanate. Alternatively, the isothiocyanate can be obtained by treatment of the amine H₂N(CH₂)ₙR¹ with carbon disulphide and a carbodiimide e.g. cyclohexylcarbodiimide or the like in an aprotic solvent such as DCM or THF.

Compounds of the invention wherein both L¹ and L² are S may be prepared from suitably substituted 2-cyanoaniline by reaction with CS₂ in pyridine followed by introduction of the N-substituent effected for instance by way of a reductive amination reaction using a desired aldehyde and a suitable reducing agent like NaBH₄ or similar.

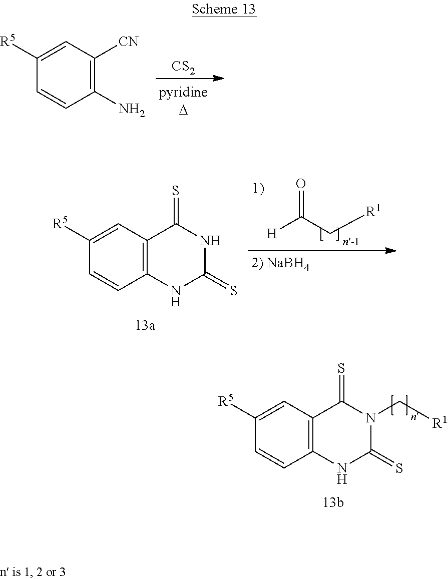

Conversion of the ring amide moieties to thioamides can be effected by thionation, using for instance Lawesson's reagent in an organic solvent such as THF or toluene typically at elevated temperature, thus providing the desired thioamide derivative.

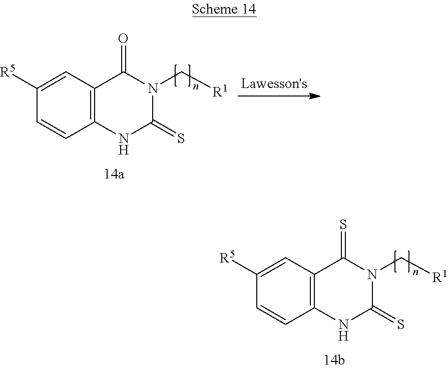

### DETAILED DESCRIPTION OF REPRESENTATIVE EMBODIMENTS

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

In general, the names of compounds used in this application are generated using ChemDraw Ultra 12.0. In addition, if the stereochemistry of a structure or a portion of a structure is not indicated with for example bold or dashed lines, the structure or portion of that structure is to be interpreted as encompassing all stereoisomers of it.

### Amine 1

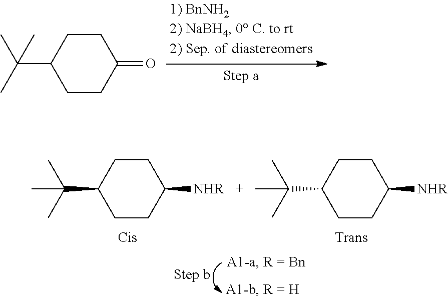

### Step a) Cis and trans-4-tert.butylcyclohexylbenzylamine (A1-a cis & A1-a trans)

To a solution of 4-tert-butylcyclohexanone (20 g, 0.13 mol) and benzylamine (16.6 g, 0.11 mol) in MeOH (160 mL), dried molecular sieves (4 Å, 10 g) was added and the mixture was stirred at room temperature for 16 h. Then, NaBH₄ (8.41 g, 0.23 mol) was added and the mixture was stirred for 6 h, then cooled to 0° C. followed by addition of water (20 mL). The solvent was removed under reduced pressure and a cold saturated aqueous solution of NaHCO₃ (40 mL) was added till pH 8 was attained. The mixture was extracted with EtOAc (3×50 mL), the combined organics was dried over sodium sulphate and concentrated. The diastereomers were separated by flash chromatography on silica using a gradient elution of 0-10% methanol/ammonia in dichloromethane, with the eluent held at 2% methanol/ammonia until the first diastereomer had eluted. In the cases investigated, the cis-product eluted before the trans-product.

Cis: 6.6 g, 21%,

¹H NMR (CDCl₃, 400 MHz) δ 7.40-7.20 (5H, m), 3.78 (2H, s), 2.90-2.86 (1H, m), 1.90-1.86 (2H, m), 1.60-1.30 (6H, m), 1.05-1.01 (1H, m), 0.86 (9H, s). MS: m/z 246 [M+1]⁺.

Trans: 18 g, 56%,

¹H NMR (MeOD, 300 MHz) δ 7.33-7.20 (5H, m), 3.75 (2H, s), 3.30-3.28 (1H, m), 2.40-2.35 (1H, m), 2.04-1.9 (2H, m), 1.82-1.80 (2H, m), 1.11-0.97 (4H, m), 0.84 (9H, s). MS: m/z 246 (M⁺+1).

### Step b) Cis-4-tert-butylcyclohexylamine (A1-b cis)

10% Pd on carbon (2.5 g) was added to a solution purged with N₂ (g), of cis-4-tert.butylcyclohexylbenzylamine (5 g, 0.02 mol) in THF (40 mL). The above mixture was hydrogenated for 16 h under atmospheric pressure, then filtered through a Celite bed and the filtrate was concentrated under reduced pressure. To the crude, 3M solution of HCl in diethylether (10 mL) was added followed by addition of EtOAc (25 mL). The resulting white solid was filtered and washed with EtOAc, which gave the hydrochloride of the title compound (1.9 g, 49%).

¹H NMR (DMSO-d₆, 400 MHz) δ 2.99 (1H, m), 2.49-2.44 (1H, m), 1.78 (2H, m), 1.67 (2H, m), 1.02-0.88 (4H, m), 0.81 (9H, s).

### Trans-4-tert-butylcyclohexylamine (A1-b trans)

The trans isomer (15 g, 0.06 mol) was taken through the same procedure as described for the cis-isomer, which gave the hydrochloride of the title compound (5.7 g, 49%).

¹H NMR (DMSO-d₆, 400 MHz) δ 3.56 (1H, m), 2.85-2.83 (1H, m), 1.98-1.95 (2H, m), 1.75-1.71 (2H, m), 1.32-1.21 (2H m), 1.05-0.90 (2H m), 0.80 (9H, m).

### Amine 2

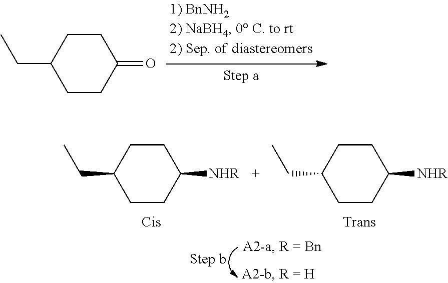

### Step a) Cis and trans-4-ethylcyclohexylamine (A2-a cis & trans)

4-Ethylcyclohexanone (10 g, 0.07 mol) and benzylamine (8.98 g, 0.06 mol) were reacted according to the procedure described in Amine 1 step a, which gave the title compounds. Cis: 3.1 g, 18%, trans: 6.4 g, 37%. MS: m/z 218 (M⁺+1).

¹H NMR (trans) (DMSO-d₆, 400 MHz) δ 7.40-7.15 (5H, m), 3.70 (2H, s), 2.30-2.20 (1H, m), 1.98-1.90 (2H, m), 1.81 (1H, brs), 1.69-1.67 (2H, m), 1.20-0.90 (4H, m), 0.8-0.6 (2H, m & 3H, t, J=8 Hz).

### Step b) Cis-4-ethylcyclohexylamine (A2-b cis & trans)

Cis-4-ethylcyclohexylbenzylamine (3 g, 13.8 mmol) and trans-4-ethylcyclohexylbenzylamine (3 g, 13.8 mmol) were each debenzylated according to the procedure described in Amine 1 step b, which gave the hydrochlorides of title compounds. Cis: 1 g, 44.3%, trans: 1.2 g, 53%.

¹H NMR (trans) (DMSO-d₆, 400 MHz) δ 2.50-2.40 (1H, m), 1.70-1.65 (4H, m), 1.35 (2H, m), 1.20-1.01 (3H, m), 0.9-0.8 (5H, m).

### Amine 3

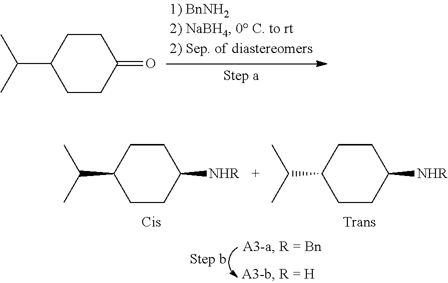

### Step a) Cis and trans-4-isopropylcyclohexylamine (A3-a cis & trans)

4-Isopropylcyclohexanone (10 g, 0.07 mol) and benzylamine (8.98 g, 0.06 mol) were reacted according to the procedure described in Amine 1 step a, which gave the title compounds. Cis: 3.6 g, 22%, MS: m/z 232 (M⁺+1).

¹H NMR (DMSO-d₆, 400 MHz): δ 7.40-7.15 (5H, m), 3.66 (2H, s), 2.66 (1H, s), 1.84-1.81 (1H, m), 1.63 (3H, m), 1.5-1.3 (6H, m), 0.80 (6H, d, J=8 Hz).

Trans: 6.8 g, 42%. MS: m/z 232 (M⁺+1).

¹H NMR (DMSO-d₆, 400 MHz) δ 7.32-7.16 (5H, m), 3.69 (2H, s), 2.26 (1H, m), 1.91 (2H, m), 1.63 (2H, m), 1.35 (1H, m), 1.02-0.85 (5H, m), 0.80 (6H, d, J=8 Hz).

### Step b) Cis-4-ethylcyclohexylamine hydrochloride salt (A3-b cis & trans)

Cis-4-isopropylcyclohexylbenzylamine (3 g, 13.0 mmol) and trans-4-isopropylcyclohexylbenzylamine (3 g, 13.0 mmol) were each debenzylated according to the procedure described in Amine 1 step b, which gave the hydrochlorides of title compounds.

Cis: 1.2 g, 51%, trans: 1.4 g, 60%.

¹H NMR (trans) (DMSO-d₆, 400 MHz) δ 1.93 (1H, m), 1.70-1.60 (3H, m), 1.50-1.20 (5H, m), 1.10-0.90 (2H, m), 0.9-0.8 (6H, m).

### Amine 4

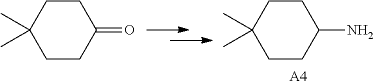

### 4,4-Dimethylcyclohexanamine (A4)

The title compound was prepared from 4,4-dimethylcyclohexanone according to the method described for the preparation of Amine 1.

¹H NMR (500 MHz, CDCl₃) δ 2.58 (m, 1H), 1.64 (m, 2H), 1.39-1.18 (m, 8H), 0.90 (s, 1H).

### Amine 5

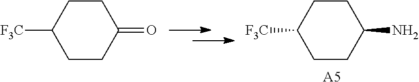

### (1R,4R)-4-(trifluoromethyl)cyclohexanamine (A5)

The title compound was prepared from 4-(trifluoromethyl)cyclohexan-1-one according to the method described for the preparation of Amine 1.

¹H NMR (500 MHz, CDCl₃) δ 2.65 (m, 1H), 1.94 (m, 5H), 1.40 (br.s, 2H), 1.36 (m, 2H), 1.09 (m, 2H).

### Amine 6

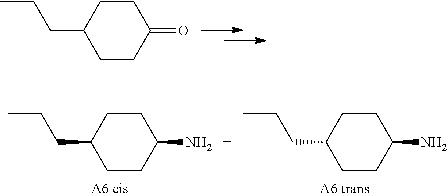

### (1R,4S)-4-propylcyclohexanamine & (1 S,4R)-4-propylcyclohexanamine (A6 cis & A6 trans)

The title compound was prepared from 4-propylcyclohexanone according to the method described for the preparation of Amine 1.

¹H NMR cis (500 MHz, CDCl₃) δ 2.94 (m, 1H), 1.55 (m, 2H), 1.50-1.21 (m, 13H), 0.88 (t, J=7.2 Hz, 3H).

### Amine 7

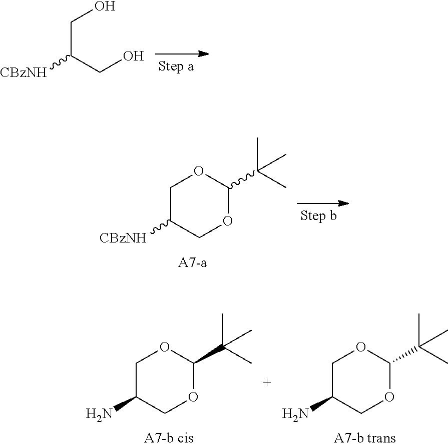

### Step a) Benzyl (2-(tert-butyl)-1,3-dioxan-5-yl)carbamate

A mixture of benzyl (1,3-dihydroxypropan-2-yl)carbamate (1.12 g, 4.98 mmol), trimethylacetaldehyde (1.03 mL, 9.48 mmol), toluenesulphonic acid monohydrate (52 mg, 0.27 mmol) and anhydrous magnesium sulphate (2.4 g, 20 mmol) in anhydrous THF (15 mL) was heated under reflux overnight. The mixture was cooled, treated with aqueous NaHCO₃ (20 mL) and stirred until effervescence ceased. The phases were separated and the aqueous phase was extracted with diethyl ether (3×20 mL). The combined organic phases were washed with water and brine, dried (MgSO₄), filtered and concentrated. The afforded oil was triturated with petroleum ether 40-60 and the resulting suspension was extracted with petroleum ether (3×50 mL) and filtered. The filtrate was concentrated, which gave the title compound as a ˜1:1 mixture of cis and trans isomers. The mixture was used in the next step without further purification.

¹H NMR (500 MHz, CDCl₃) δ 7.37 (m, 5H), 5.66 (d, J=9.2 Hz, 0.5H), 5.17-5.10 (m, 2.5H), 4.56 (m, 0.5H), 4.22 (m, 1H), 4.16 (s, 0.5H), 4.00 (m, 1.5H), 3.89 (m, 1H), 3.64 (m, 0.5H), 3.30 (m, 1H), 0.93 (s, 9H).

### Step b) (2R,5R)-2-(tert-butyl)-1,3-dioxan-5-amine & (2S,5S)-2-(tert-butyl)-1,3-dioxan-5-amine (Cis) (A7-b trans & A7-b cis)

A vessel containing a solution of the benzyl (2-(tert-butyl)-1,3-dioxan-5-yl)carbamate (1.03 g, 3.50 mmol) in MeOH (10 mL) was purged with three vacuum/argon cycles. Palladium on carbon (0.120 g) was added and the vessel was purged three times with vacuum/argon and then three times with vacuum/hydrogen cycles. The mixture was stirred at room temperature overnight, then filtered through a plug of Celite and concentrated. The diastereomers were separated by flash chromatography on silica eluted with a gradient of 0-10% MeOH/ammonia in DCM to give first the trans product, (2r,5r)-2-(tert-butyl)-1,3-dioxan-5-amine (0.218 g, 1.37 mmol, 39%) and then the cis product (2s,5s)-2-(tert-butyl)-1,3-dioxan-5-amine (0.211 g, 1.33 mmol, 38%).

¹H NMR trans (500 MHz, CDCl₃) δ 4.13 (ddd, J=9.8, 4.9, 1.4 Hz, 2H), 3.99 (s, 1H), 3.19 (ddd appearing as td, J=10.3, 1.3 Hz), 3.01 (m, 1H), 1.00 (br.s, 2H), 0.91 (s, 9H).

¹H NMR cis (500 MHz, CDCl₃) δ 4.12 (s, 1H), 3.90 (dd, J=10.5, 1.7 Hz, 2H), 3.87 (dd, J=10.5, 1.7 Hz, 2H), 2.65 (m, 1H), 1.83 (br.s, 2H), 0.92 (s, 9H).

### Amine 8

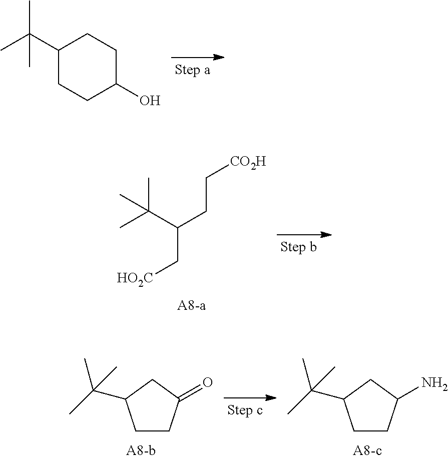

### Step a) 3-(tert-butyl)hexanedioic acid (A8-a)

Sodium nitrite (17.0 g, 250 mmol) was added at 0° C. in small portions over 90 minutes to a solution of 4-tert-butylcyclohexanol (10.0 g, 64 mmol) in TFA (100 mL). The suspension was stirred at room temperature overnight and then concentrated. The residue was poured onto ice, treated with aqueous NaHCO₃ (500 mL) and then made basic by addition of solid NaOH. The aqueous solution was washed with DCM (3×200 mL), acidified to pH <1 with concentrated HCl and extracted with EtOAc (3×300 mL). The combined organic phases were dried (MgSO₄), filtered and concentrated, which gave the title compound (13.05 g, ˜100%) which was used in the next step without further purification.

¹H NMR (500 MHz, CDCl₃) δ 9.63 (br.s, 2H), 2.56 (dd, J=16.3 and 3.9 Hz, 1H), 2.50-2.39 (m, 2H), 2.12 (dd, J=16.3 and 8.0 Hz, 1H), 1.98 (m, 1H), 1.74 (m, 1H), 1.44 (m, 1H), 0.93 (s, 9H).

### Step b) 3-(tert-butyl)cyclopentanone (A8-b)

Crude diacid A8-a (8.33 g, 41 mmol) and solid sodium carbonate (0.22 g, 2.0 mmol) were heated together to 240° C. in a Kugelrohr distillation apparatus for 1 hour. The title compound distilled from the reaction mixture and was collected as an oil (1.98 g, 34%).

¹H NMR (500 MHz, CDCl₃) δ 2.38 (dd, J=8.1, 1.1 Hz), 2.29-2.17 (m, 2H), 2.07-1.94 (m, 2H), 1.61 (m, 1H), 0.93 (s, 9H).

### Step c) 3-(tert-butyl)cyclopentanamine (A8-c)

The title compound (mix of cis and trans) was prepared by treatment of ketone A8-b according to the method described in A1 steps a and b.

¹H NMR (500 MHz, CDCl₃) δ 3.56-3.24 (m, 1H), 2.00-1.84 (m, 2H), 1.73 (m, 1H), 1.59 (m, 1H), 1.51-1.44 (m, 3H), 1.37-1.22 (m, 2H), 0.86 (s, 5.5H), 0.85 (s, 3.5H).

### Amine 9

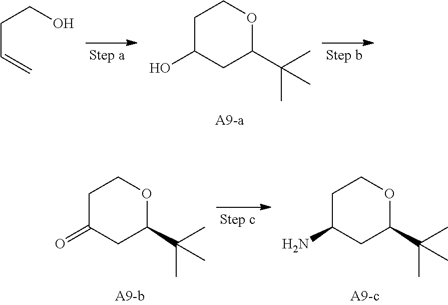

### Step a) 2-methyltetrahydro-2H-pyran-4-ol (A9-a)

TFA (5 mL) was added to a cooled solution (0° C.) of trimethylacetaldehyde (2.2 mL, 20 mmol) and 3-buten-1-ol (1.7 mL, 20 mmol) in CH₂Cl₂ (15 mL). The mixture was allowed to attain to room temperature and stirred overnight. The mixture was concentrated, redissolved in MeOH (20 mL) and cooled to 0° C. Solid potassium carbonate (3.6 g) was added portionwise and the mixture was stirred at room temperature for 5 hours, then concentrated. The residue was extracted with Et₂O (100 mL) and concentrated which gave the title compound (2.87 g, 91%), which was used without further purification

¹H NMR (500 MHz, CDCl₃) δ 4.04 (ddd, J=11.7, 5.0, 1.6 Hz, 1H), 3.77 (m, 1H), 3.36 (td, J=12.2, 2.1 Hz, 1H), 2.88 (dd, J=11.4, 1.7 Hz, 1H), 1.97 (m, 1H), 1.88 (m, 1H), 1.76 (br.s, 1H), 1.48 (m, 1H), 1.22 (m, 1H), 0.92 (s, 9H).

### Step b) (R)-2-(tert-butyl)dihydro-2H-pyran-4(3H)-one (A9-b)

Dess-Martin Periodinane (4.04 g, 9.53 mmol) was added to a solution of the alcohol A9-a (1.01 g, 6.37 mmol) in CH₂Cl₂ (125 mL) and the mixture was stirred at room temperature for 3 days. Saturated aqueous NaHCO₃ (100 mL) and 20% aqueous sodium thiosulphate (100 mL) were then added and the mixture was vigorously stirred for 1 hour. The phases were separated and the aqueous phase was extracted with CH₂Cl₂ (2×100 mL). The combined organic extracts were concentrated. The afforded residue was purified by flash chromatography (0-100% Et₂O/hexane) which gave the title compound, (0.648 g, 65%).

¹H NMR (500 MHz, CDCl₃) δ 4.32 (ddd, J=11.4, 7.6, 1.0 Hz, 1H), 3.61 (ddd, J=14.1, 11.4, 2.7 Hz, 1H), 3.20 (dd, J=11.5, 2.7 Hz, 1H), 2.58 (m, 1H), 2.42-2.29 (m, 3H), 0.95 (s, 9H).

### Step c) (2R,4S)-2-(tert-butyl)tetrahydro-2H-pyran-4-amine (A9-c)

The title compound was prepared by treatment of ketone A9-b according to the method described in A1 steps a and b.

¹H NMR (500 MHz, CDCl₃) δ 3.56-3.24 (m, 1H), 2.00-1.84 (m, 2H), 1.73 (m, 1H), 1.59 (m, 1H), 1.51-1.44 (m, 3H), 1.37-1.22 (m, 2H), 0.86 (s, 5.5H), 0.85 (s, 3.5H).

### Amine 10

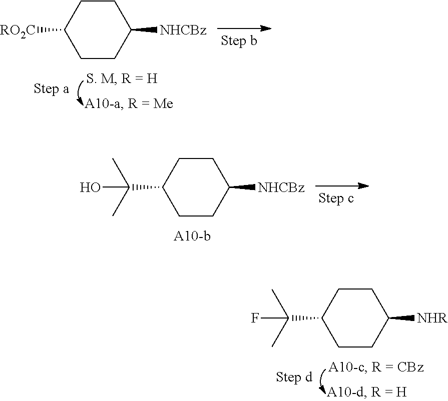

### Step a) (1r,4r)-methyl 4-(((benzyloxy)carbonyl)amino)cyclohexanecarboxylate (A10-a)

A suspension of trans-4-(carbobenzoxyamino)cyclohexanecarboxylic acid (8.09 g, 29.2 mmol) and freshly-ground potassium carbonate (4.24 g, 30.7 mmol) in DMF (140 mL) was stirred at room temperature for 40 minutes then cooled to 0° C. Iodomethane (1.90 mL, 30.5 mmol) was added dropwise. The mixture was stirred at room temperature overnight then concentrated and partitioned between water (100 mL) and EtOAc (3×100 mL). The combined organic phases were dried (MgSO₄), filtered and concentrated which gave the title compound, (7.72 g, 91%).

¹H NMR (500 MHz, CDCl₃) δ 7.35 (m, 5H), 5.11 (s, 2H), 4.64 (br.s, 1H), 3.69 (s, 3H), 3.52 (m, 1H), 2.25 (tt, J=12.2, 3.6 Hz, 1H), 2.11 (br.d, J=10.8 Hz, 2H), 2.04 (br.d, J=13.2 Hz, 2H), 1.56 (br.q, J=12.1 Hz, 2H), 1.16 (qd, J=12.8, 3.4 Hz, 2H).

### Step b) benzyl ((1r,4r)-4-(2-hydroxypropan-2-yl)cyclohexyl)carbamate (A10-b)

A 3M solution of MeMgBr in Et₂O (10.5 mL, 31.5 mmol) was added dropwise at 0° C. to a solution of the ester A10-a (3.00 g, 10.3 mmol) in Et₂O (180 mL). The suspension was stirred at 0° C. for 90 minutes and then at room temperature for 21 hours. Saturated aqueous NH₄Cl (100 mL) was added to the reaction mixture was treated and the phases were separated. The aqueous phase was extracted with Et₂O (2×100 mL) and the combined organic phases were washed with brine, dried (MgSO₄), filtered and concentrated. The residue was purified by flash chromatography (0-100% EtOAc/hexane) which gave the title compound (1.60 g, 53%).

¹H NMR (500 MHz, CDCl₃) 7.35 (m, 5H), 5.11 (s, 2H), 4.62 (br.s, 1H), 3.46 (m, 1H), 2.12 (br.d, J=11.0 Hz, 2H), 1.89 (br.d, J=12.0 Hz, 2H), 1.31-1.09 (m, 11H).

### Step c) benzyl ((1r,4r)-4-(2-fluoropropan-2-yl)cyclohexyl)carbamate (A10-c)

A suspension of the alcohol A10-b (0.585 g, 2.01 mmol) in CH₂Cl₂ at −78° C. was treated with DBU (0.45 mL, 3.0 mmol) and XtalFluor-E (0.684 g, 2.99 mmol). The resulting pale yellow solution was stirred at −78° C. for 1 hour, then at room temperature overnight. The resulting solution was treated with saturated aqueous NaHCO₃ (12 mL) and vigorously stirred for 15 minutes. The phases were separated, the aqueous phase was extracted with CH₂Cl₂ and the organic phases were filtered through plugs of MgSO₄ and silica and concentrated. Purification by flash chromatography (0-100% EtOAc/p.ether 40-60) gave the title compound as a solid (0.486 g, 82%).

¹H NMR (500 MHz, CDCl₃) δ 7.35 (m, 5H), 5.11 (s, 2H), 4.61 (br.s, 1H), 3.47 (m, 1H), 2.12 (br.d, J=11.5 Hz, 2H), 1.87 (br.d, J=12.7 Hz, 2H), 1.51 (m, 1H), 1.32 (d, J=22.1 Hz, 6H), 1.28-1.09 (m, 4H).

### Step d) (1r,4r)-4-(2-fluoropropan-2-yl)cyclohexanamine (A10-d)

A vessel containing a solution of the carbamate A10-c (0.360 g, 1.23 mmol) in EtOH (5 mL) was purged with three vacuum/argon cycles. Palladium on carbon (60 mg) was added and the vessel purged three times with vacuum/argon and then three times with vacuum/hydrogen cycles. The mixture was stirred at room temperature for three days, then filtered through a plug of Celite and concentrated, which gave the title compound (0.180 g, 92%).

¹H NMR (500 MHz, CDCl₃) δ 2.54 (m, 1H), 1.85 (m, 2H), 1.74 (m, 2H), 1.43 (m, 3H), 1.22 (d, J=22.1 Hz, 6H), 1.10-0.98 (m, 4H).

### Example 1

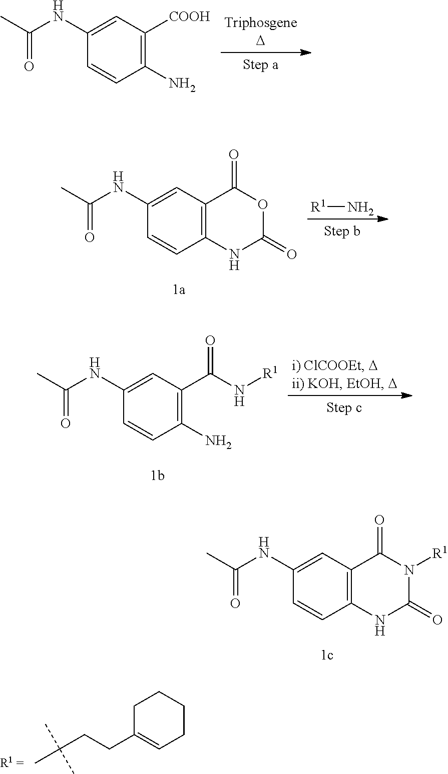

### Step a): N-(2,4-Dioxo-1,4-dihydro-2H-benzo[d][1,3]oxazin-6-yl)-acetamide (1a)

triphosgene (3.8 g, 1.28 mmol) was added to a solution of 5-acetamido-2-amino-benzoic-acid (5 g, 25.7 mmol) in 1,4-dioxane (100 mL) and the solution was heated at 110° C. for 6 h. The solution was then cooled to room temperature whereafter a saturated aqueous solution of NaHCO₃ (20 mL) was added. The mixture was filtered and the filtered solid was washed with water followed by hexanes. The solid was dried at 60° C. under vacuum to afford the title compound as a solid (5 g, 89%).

¹H NMR (DMSO-d₆, 400 MHz) δ 11.66 (1H, s), 10.17 (1H, s), 8.24 (1H, d, J=2.4 Hz), 7.80 (1H, dd, J=8.8, 2.4 Hz), 7.10 (1H, d, J=8.8 Hz), 2.04 (3H, s). MS: m/z 221 [M+1]⁺.

### Step b) 5-Acetamido-2-amino-N-(2-(cyclohex-1-en-1-yl)ethyl)benzamide (1b)

DMAP (0.5 mmol) was added to a solution of cyclohexenyl ethylamine (0.087 g, 0.7 mmol) and the isatoic anhydride 1a (0.1 g, 0.45 mmol) was dissolved in DMF (10 mL) followed by addition of DMAP (0.5 mmol). The solution was stirred at room temperature for 3 h. After removal of solvent under reduced pressure water was added to the crude and extracted with EtOAc (3×10 mL). The combined organics were dried (Na₂SO₄), filtered and concentrated. The crude material was taken to the next step without further purification.

### Step c) N-[3-(2-Cyclohex-1-enyl-ethyl)-2,4-dioxo-1,2,3,4-tetrahydro-quinazolin-6-yl]-acetamide (1c)

The crude amide from step 2 (3.1 mmol) and ethylchloroformate (4 mL) was heated at 90° C. for 1.5 h. The solvent was removed under reduced pressure and the crude was dissolved in EtOH (40 mL) followed by addition of KOH (0.35 g, 6.3 mmol). The mixture was heated at 85° C. for 2 h. The solvent was removed under reduced pressure followed by addition of water (10 mL) and extraction with EtOAc (3×20 mL). The combined organics was washed with 10% aqueous solution of acetic acid till pH 6. This was followed by extraction with EtOAc (3×20 mL) and the combined organics dried over sodium sulphate and the solvent concentrated. The crude was purified by flash column chromatography on silica gel eluted with MeOH in CHCl₃ which gave the title compound (0.045 g, 30%) over two steps.

¹H NMR (DMSO-d₆, 400 MHz) δ 11.33 (1H, s), 10.09 (1H, s), 8.21 (1H, J=2.4 Hz), 7.78 (1H, dd, J=8.8 Hz, 2.4 Hz), 7.11 (1H, d, J=8.8 Hz), 5.29 (1H, m), 3.97-3.93 (2H, m), 1.19-2.14 (2H, m), 2.03 (3H, s), 1.98 (2H, m), 1.85 (2H, m), 1.58-1.52 (4H, m). MS: m/z 326 (M⁺−1)⁻.

### Example 2

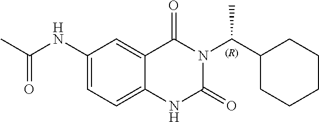

### (R)—N-(3-(1-cyclohexylethyl)-2,4-dioxo-1,2,3,4-tetrahydroquinazolin-6-yl)acetamide (2)

The isatoic anhydride 1a (0.2 g, 0.9 mmol) and (R)-1-cyclohexylethan-1-amine (0.11 g, 0.9 mmol) were reacted according to the procedure described in Example 1 steps b and c, which gave the title compound (0.1 g, 34%) over two steps.

¹H NMR (DMSO-d₆, 400 MHz): δ 11.50-11.10 (1H, br m), 10.08 (1H, s), 8.21 (1H, d, J=2.4 Hz), 7.77-7.75 (1H, m), 7.09 (1H, d, J=8.4 Hz), 4.75-4.55 (1H, m), 2.18-2.09 (1H, m), 2.04 (3H, s), 1.89-1.86 (1H, m), 1.73-1.70 (1H, m), 1.59-1.57 (2H, m), 1.40-1.34 (4H, m), 1.35-1.20 (2H, m), 1.17-1.11 (2H, m), 0.91-0.88 (2H, m). MS: m/z 328 [M−1]⁻.

### Example 3

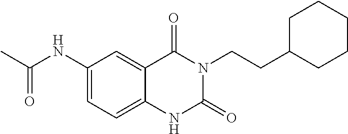 [SMILES: CC(=O)Nc1ccc2[nH]c(=O)n(CCC3CCCCC3)c(=O)c2c1]

### N-[3-(2-Cyclohexylethyl)-2,4-dioxo-1,2,3,4-tetrahydroquinazolin-6-yl]acetamide (3)

Isatoic anhydride 1a (1.0 g, 4.5 mmol) and cyclohexyl ethylamine (0.89 g, 7 mmol) were reacted according to the procedure described in Example 1 steps b and c, which gave the title compound (0.8 g, 54%) over two steps.

¹H NMR (DMSO-d₆, 400 MHz): δ 11.35 (1H, s), 10.09 (1H, s), 8.21 (1H, d, J=2 Hz), 7.78 (1H, dd, J=8.8, 2 Hz), 7.11 (1H, d, J=8.8 Hz), 3.92-3.88 (2H, m), 2.04 (3H, s), 1.76-1.72 (3H, m), 1.66-1.64 (3H, m), 1.45-1.42 (2H, m), 1.33-1.23 (3H, m), 1.22-1.18 (2H, m). MS m/z 328 [M−1]⁻.

### Example 4

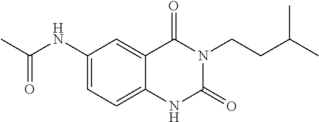 [SMILES: CC(=O)Nc1ccc2[nH]c(=O)n(CCC(C)C)c(=O)c2c1]

### N-(3-Isopentyl-2,4-dioxo-1,2,3,4-tetrahydroquinazolin-6-yl)acetamide (4)

The isatoic anhydride 1a (0.3 g, 1.3 mmol) and isopentyl amine (0.11 g, 1.3 mmol) were reacted according to the procedure described in Example 1 steps b and c, which gave the title compound (0.15 g, 38%) over two steps.

¹H NMR (DMSO-d₆, 400 MHz): δ 11.34 (1H, s), 10.09 (1H, s), 8.21 (1H, d, J=2.4 Hz), 7.77 (1H, dd, J=8.8, 2.4 Hz), 7.10 (1H, d, J=8.8 Hz), 3.90-3.86 (1H, m), 2.03 (3H, s), 1.60-1.53 (1H, m), 1.45-1.40 (2H, m), 0.91-0.90 (6H, d, J=6.4 Hz). MS m/z 290 [M+1]⁺.

### Example 5

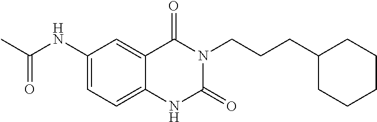 [SMILES: CC(=O)Nc1ccc2[nH]c(=O)n(CCCC3CCCCC3)c(=O)c2c1]

### N-[3-(3-Cyclohexyl-propyl)-2,4-dioxo-1,2,3,4-tetrahydro-quinazolin-6-yl]-acetamide (5)

The isatoic anhydride 1a (0.1 g, 0.45 mmol) and cyclohexyl ethylamine (0.098 g, 0.7 mmol) were reacted according to the procedure described in Example 1 steps b and c, which gave the title compound (0.045 g, 29%) over two steps.

¹H NMR (DMSO-d₆, 400 MHz): δ 11.34 (1H, s), 10.10 (1H, s), 8.22 (1H, d, J=2 Hz), 7.78 (1H, dd, J=8.8, 2 Hz), 7.12 (1H, d, J=8.8 Hz), 3.86-3.82 (1H, m), 2.04 (3H, s), 1.70-1.5 (7H, m), 1.22-1.13 (6H, m), 0.9-0.8 (2H, m). MS m/z 342 [M−1]⁻.

### Example 6

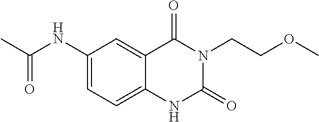 [SMILES: COCCn1c(=O)[nH]c2ccc(NC(C)=O)cc2c1=O]

### N-(3-(2-methoxyethyl)-2,4-dioxo-1,2,3,4-tetrahydroquinazolin-6-yl)acetamide (6)

The isatoic anhydride 1a (0.8 g, 3.6 mmol) and 2-methoxyethyl amine (0.41 g, 5.4 mmol) were reacted according to the procedure described in Example 1 steps b and c, which gave the title compound (0.28 g, 28%) over two steps.

¹H NMR (DMSO-d₆, 400 MHz): δ 11.35 (1H, s), 10.09 (1H, s), 8.22 (1H, d, J=2.4 Hz), 7.78 (1H, dd, J=8.8, 2.4 Hz), 7.12 (1H, d, J=8.8 Hz), 4.07 (2H, t, J=6), 3.51 (2H, t, J=6), 3.24 (3H, s), 2.04 (3H, s). MS m/z 278 [M+1]⁺.

### Example 7

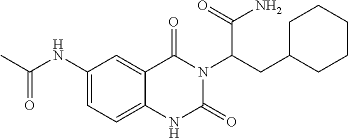 [SMILES: CC(=O)Nc1ccc2[nH]c(=O)n(C(CC3CCCCC3)C(N)=O)c(=O)c2c1]

### 2-(6-Acetamido-2,4-dioxo-1,4-dihydroquinazolin-3(2H)-yl)-3-cyclohexylpropanamide (7)

The isatoic anhydride 1a (0.1 g, 0.45 mmol) and 2-amino-3-cyclohexylpropanamide (0.13 g, 0.67 mmol) were reacted according to the procedure described in Example 1 steps b and c, which gave the title compound (0.025 g, 15%) over two steps.

¹H NMR (DMSO-d₆, 400 MHz): δ 11.33 (1H, br s), 10.10 (1H, s), 8.21 (1H, d, J=2.4 Hz), 7.78 (1H, dd, J=8.8, 2.4 Hz), 7.31 (1H, s), 7.12 (1H, d, J=8.8 Hz), 6.90 (1H, s), 5.28-5.25 (1H, m), 2.04 (3H, s), 2.0-1.80 (3H, m), 1.62-1.49 (4H, m), 1.19-0.09 (4H, m), 0.89-0.79 (2H, m). MS m/z 371 [M−1]⁻.

### Example 8

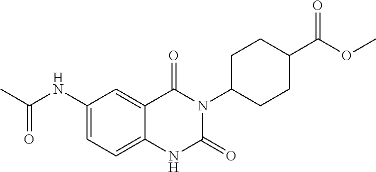 [SMILES: COC(=O)C1CCC(n2c(=O)[nH]c3ccc(NC(C)=O)cc3c2=O)CC1]

### Methyl 4-(6-acetamido-2,4-dioxo-1,4-dihydroquinazolin-3(2H)-yl)cyclohexane-1-carboxylate (8)

The isatoic anhydride 1a (1.0 g, 4.5 mmol) was reacted with methyl 4-aminocyclohexane-1-carboxylate hydrochloride (1.31 g, 6.8 mmol) according to the procedure described in Example 1 step b. The obtained crude (1 g) was dissolved in THF (10 mL) and, 1,1′-carbonyldiimidazole (1.45 g, 9 mmol) was added and the solution was heated at 90° C. for 48 h. The solvent was removed under reduced pressure followed by addition of water (10 mL) and extraction with EtOAc (3×20 mL). The solvent was removed under reduced pressure and the crude was purified by flash column chromatography on silica gel, which gave the title compound (0.7 g, 43% after 2 steps) as a solid.

¹H NMR (DMSO-d₆, 400 MHz): δ 11.27 & 11.19 (1H, s each), 10.08 & 10.07 (1H, s each), 8.18 (1H, d, J=2 Hz), 7.79-7.73 (1H, m), 7.07 (1H, d, J=8.8 Hz), 4.76-4.69 (1H, m), 3.66 & 3.60 (3H, s each), 2.69 (1H, m), 2.49-2.40 (2H, m), 2.18-2.15 (2H, m), 2.02 (3H, s), 1.58-1.22 (4H, m). MS: m/z 360 [M+1]⁺.

### Example 9

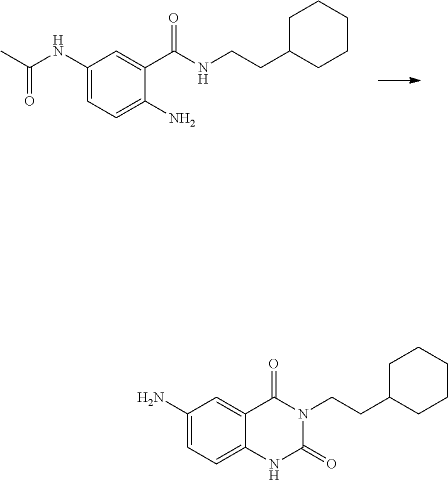 [SMILES: CC(=O)Nc1ccc(N)c(C(=O)NCCC2CCCCC2)c1 ; Nc1ccc2[nH]c(=O)n(CCC3CCCCC3)c(=O)c2c1]

### 6-Amino-3-(2-cyclohexylethyl)quinazoline-2,4(1H,3H)-dione (9)

5-Acetylamino-2-amino-N-(2-cyclohexyl-ethyl)-benzamide (0.1 g, 0.33 mmol), was taken in ethylchloroformate (0.4 mL) and heated to 90° C. for 1.5 h. The solvent was removed under reduced pressure and the crude was dissolved in EtOH (2 mL) followed by addition of KOH (0.15 g, 2.6 mmol) and heating at 85° C. for 16 h. The solvent was removed under reduced pressure followed by addition of water (10 mL) and extraction with EtOAc (3×10 mL). The combined organics was dried (Na₂SO₄), filtered and concentrated. The crude was purified by flash column chromatography on silica gel, which gave title compound as a solid (0.035 g, 36%).

¹H NMR (DMSO-d₆, 400 MHz): δ 10.98 (1H, s), 7.08 (1H, d, J=2 Hz), 6.95-6.88 (2H, m), 5.16 (2H, s), 3.89-3.85 (2H, m), 1.74-1.58 (7H, m), 1.44-1.25 (4H, m), 0.9-0.8 (2H, m). MS: m/z 288 [M+1]⁺.

### Example 10

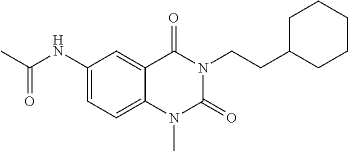 [SMILES: CC(=O)Nc1ccc2c(c1)c(=O)n(CCC1CCCCC1)c(=O)n2C]

### N-[3-(2-Cyclohexyl-ethyl)-1-methyl-2,4-dioxo-1,2,3,4-tetrahydro-quinazolin-6-yl]-acetamide (10)

A suspension of NaH (60% in mineral oil, 7 mg, 0.17 mmol) in DMA (0.5 mL) was cooled to 0° C. followed by addition of N-[3-(2-cyclohexyl-ethyl)-2,4-dioxo-1,2,3,4-tetrahydro-quinazolin-6-yl]-acetamide (0.05 g, 0.15 mmol) and stirring at 0° C. for 30 min. This was followed by addition of methyl iodide (0.009 mL, 0.15 mmol) at 0° C. and kept at the same temperature for 5 min then stirred at room temperature for 5 min. Ice-water was added to the reaction mixture followed by neutralization with 1.5 N HCl till pH 6. This was followed by extraction with EtOAc (2×5 mL) and drying over sodium sulphate. The organics were removed under reduced pressure followed by purification of the crude by flash column chromatography on silica gel (230-400 mesh, 2.5% MeOH in CHCl₃) which gave the title compound (0.035 g, 68%) as a solid.

¹H NMR (DMSO-d₆, 400 MHz): δ 10.16 (1H, s), 8.30 (1H, d, J=2.4 Hz), 7.91 (1H, dd, J=9 Hz), 3.70-3.92 (2H, m), 3.48 (3H, s), 2.05 (3H, s), 1.95-1.75 (2H, m), 1.72-1.59 (5H, m), 1.61-1.59 (2H, m), 1.47-1.28 (4H, m), 1.0-0.8 (2H, m). MS: m/z 344 [M+1]⁺.

### Example 11

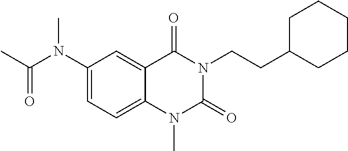 [SMILES: CC(=O)N(C)c1ccc2c(c1)c(=O)n(CCC1CCCCC1)c(=O)n2C]

### N-(3-(2-cyclohexylethyl)-1-methyl-2,4-dioxo-1,2,3,4-tetrahydroquinazolin-6-yl)-N-methylacetamide (11)

A suspension of NaH (60% in mineral oil, 8 mg, 0.19 mmol) in DMA (0.6 mL) was cooled to 0° C. followed by addition of N-[3-(2-cyclohexyl-ethyl)-2,4-dioxo-1,2,3,4-tetrahydro-quinazolin-6-yl]-acetamide (0.05 g, 0.19 mmol). The suspension was stirred at 0° C. for 30 min, methyl iodide (0.002 mL, 0.22 mmol) was added and the stirring was continued at room temperature for 16 h. Ice-water was added to the reaction mixture followed by neutralization with 1.5N HCl till pH 6. The reaction mixture was extracted with EtOAc (2×5 mL), dried (Na₂SO₄), filtered and concentrated. The afforded crude product was purified by flash column chromatography on silica gel which gave the title compound (0.035 g, 66%) as a solid.

¹H NMR (DMSO-d₆, 400 MHz): δ 7.91 (1H, s), 7.75 (1H, br s), 7.50 (1H, d, J=8.4 Hz), 3.97-3.93 (2H, m), 3.52 (3H, s), 3.15 (3H, s), 1.76-1.64 (8H, m), 1.46-1.44 (2H, m), 1.22-1.11 (4H, m), 0.96-0.93 (2H, m). MS: m/z 358 (M⁺+1).

### Example 12

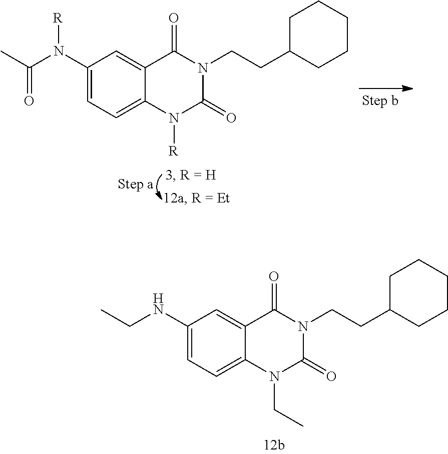

### Step a) N-(3-(2-cyclohexylethyl)-1-ethyl-2,4-dioxo-1,2,3,4-tetrahydroquinazolin-6-yl)-N-ethylacetamide (12a)

N-[3-(2-cyclohexyl-ethyl)-2,4-dioxo-1,2,3,4-tetrahydro-quinazolin-6-yl]-acetamide (0.3 g, 0.9 mmol) was alkylated with ethyl iodide (0.41 g, 2.7 mmol) according to the method described in Ex. 11 step a, which gave the title compound. MS: m/z 386 [M+1]⁺.

### Step b) 3-(2-cyclohexylethyl)-1-ethyl-6-(ethylamino)quinazoline-2,4(1H,3H)-dione (12b)

KOH (0.15 g, 2.6 mmol) was added to a solution of the crude compound from the previous step (0.2 g) in EtOH (2 mL), and the solution was heated to 90° C. for 16 h. The solvent was removed under reduced pressure, water (10 mL) was added and the mixture was extracted with EtOAc (3×10 mL). The combined organics was dried (Na₂SO₄), filtered and concentrated, and the crude was purified by flash column chromatography on silica gel, which gave the title compound (0.09 g, 29% after 2 steps).

¹H NMR (DMSO-d₆, 400 MHz): δ 7.27 (1H, d, J=9.2 Hz), 7.11 (1H, d, J=2.8 Hz), 7.06 (1H, dd, J=9.2, 2.8 Hz), 5.80-5.77 (1H, m), 4.08-4.03 (2H, m), 3.95-3.92 (2H, m), 3.06-3.03 (2H, m), 1.74-1.71 (2H, m), 1.66-1.58 (3H, m), 1.46-1.40 (2H, m), 1.25-1.15 (10H, m), 0.92-0.89 (2H, m). MS: m/z 344 (M⁺+1).

### Example 13

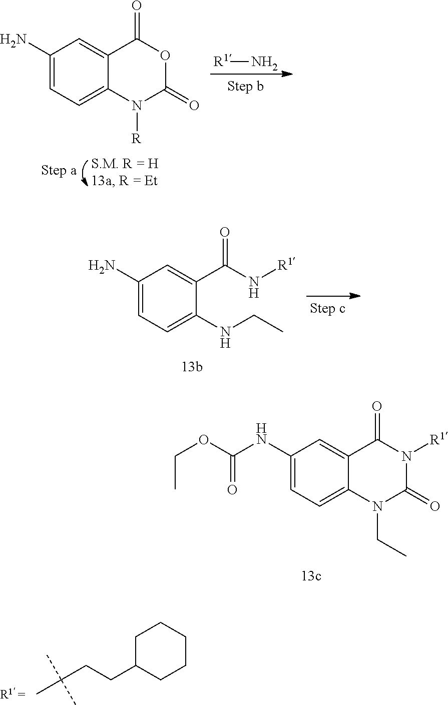

### Step a) 6-Amino-1-ethyl-2H-benzo[d][1,3]oxazine-2,4(1H)-dione (13a)

Ethyl iodide (0.8 g, 5.0 mmol) was added at 0° C. to a solution of 5-aminoisatoic anhydride (0.6 g, 3.3 mmol) in DMA (12 mL). The reaction was stirred at room temperature for 16 h, then concentrated under reduced pressure. The crude 6-amino-1-ethyl-2H-benzo[d][1,3]oxazine-2,4(1H)-dione (0.152 g) was taken to the next step without purification. MS: m/z 207 [M+1]⁺.

### Step b) 5-Amino-N-(2-cyclohexylethyl)-2-(ethylamino)benzamide (13b)

The crude material (0.15 g) from previous step was added to a solution of cyclohexylethyl amine (0.14 g, 1.1 mmol) and DMAP (20 mg) in DMF (0.75 mL). The reaction mixture was stirred at room temperature for 3 h, then concentrated under reduced pressure. Water (10 mL) was added and the mixture was extracted with EtOAc (3×10 mL). The combined organic phases were dried (Na₂SO₄), filtered and concentrated. The crude was used in the next step without further purification. MS: m/z 290 [M+1]⁺.

### Step c) Ethyl (3-(2-cyclohexylethyl)-1-ethyl-2,4-dioxo-1,2,3,4-tetrahydroquinazolin-6-yl)carbamate (13c)

The crude material from previous step (0.037 g, 0.12 mmol) was taken in ethylchloroformate (0.13 mL) and heated to 90° C. for 1.5 h. The mixture was then concentrated under reduced pressure. The afforded crude was dissolved in EtOH (1.2 mL), KOH (0.014 g, 0.25 mmol) was added and the mixture was heated at 85° C. for 2 h, then concentrated under reduced pressure. Water (5 mL) was added and the mixture was extracted with EtOAc (3×5 mL). The combined organics was dried (Na₂SO₄), filtered and concentrated and the crude was purified by flash column chromatography on silica gel, which gave the title compound (0.034 g, 75%) as a solid.

¹H NMR (DMSO-d₆, 400 MHz): δ 11.48 (1H, s), 7.71 (1H, d, J=2.4 Hz), 7.50 (1H, dd, J=8.8, 2.4 Hz), 7.17 (1H, d, J=8.8 Hz), 4.04 (2H, q, J=6.8 Hz), 3.90 (2H, t, J=7.2 Hz), 3.63 (2H, q, J=6.8 Hz), 1.76-1.73 (2H, m), 1.67-1.60 (3H, m), 1.48-1.42 (2H, m), 1.27-1.22 (7H, m), 1.04 (3H, t, J=6.8 Hz), 0.96-0.90 (2H, m). MS: m/z 388 [M+1]⁺.

### Example 14

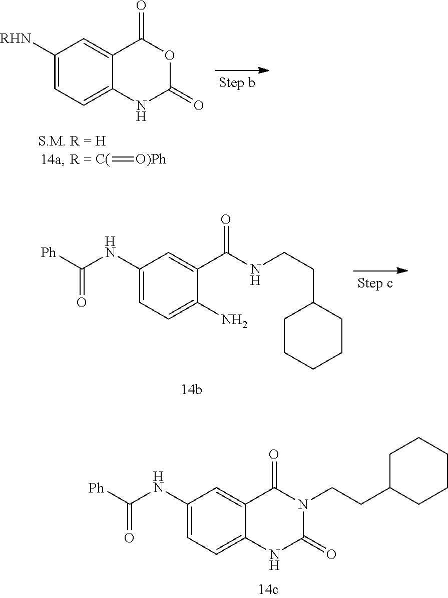

### Step a) I-(2,4-Dioxo-1,4-dihydro-2H-benzo[d][1,3]oxazin-6-yl)benzamide (14a)

HATU (0.93 g, 2.44 mmol) was added to a solution at 0° C. of benzoic acid (0.25 g, 2.04 mmol) in DMF (5 mL). The mixture was stirred for 10 min, then, 5-aminoisatoic anhydride (0.64 g, 2.04 mmol) and NMM (0.61 g, 6.12 mmol) were added and the stirring was continued at room temperature for 16 h. The solvent was removed under reduced pressure, water (10 mL) was added and the mixture was extracted with EtOAc (3×10 mL). The combined organics were dried (Na₂SO₄), filtered and concentrated. The afforded crude compound was used in next step without further purification. MS: m/z 283 [M+1]⁺.

### Step b) 2-Amino-5-benzamido-N-(2-cyclohexylethyl)benzamide (14b)

The crude material (0.18 g) from previous step was added to a solution of cyclohexylethyl amine (0.12 g, 0.9 mmol) and DMAP (16 mg) in DMF (0.8 mL). The reaction mixture was stirred at room temperature for 3 h, then concentrated under reduced pressure. Water (10 mL) was added and the mixture was extracted with EtOAc (3×10 mL). The combined organics were dried (Na₂SO₄), filtered and concentrated under reduced pressure. The afforded crude compound was taken to the next step without further purification. MS: m/z 366 [M+1]⁺.

### Step c) N-(3-(2-Cyclohexylethyl)-2,4-dioxo-1, 2,3,4-tetrahydroquinazolin-6-yl)benzamide (14c)

The crude material from previous step (0.09 g), was taken in ethylchloroformate (0.35 mL) and heated to 90° C. for 1.5 h. The solvent was removed under reduced pressure and the crude was dissolved in EtOH (3.36 mL) followed by addition of KOH (0.06 g, 1.06 mmol) and heating at 85° C. for 5 h. The solvent was removed under reduced pressure, water (5 mL) was and the mixture was extracted with EtOAc (3×5 mL). The combined organics were dried (Na₂SO₄), filtered and concentrated. The crude was purified by flash column chromatography on silica gel, which gave the title compound (0.055 g, 57%). MS: m/z 390 (M⁺−1).

### Example 15

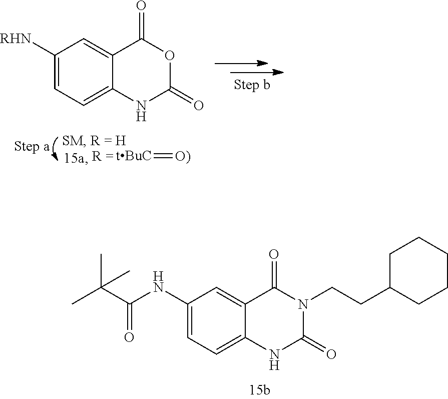

### Step a) N-(2,4-dioxo-1,4-dihydro-2H-benzo[d][1,3]oxazin-6-yl)-3,3-dimethylbutanamide (15a)

EDC×HCl (0.8 g, 4.2 mmol) was added to a solution of tert-butyl acetic acid (0.97 g, 8.4 mmol) in DMF (11 mL). The mixture was stirred at room temperature for 30 min, then, 5-aminoisatoic anhydride (0.5 g, 2.8 mmol) was added the stirring was continued at room temperature. After 16 h, additional tert-butyl acetic acid (0.65 g, 5.6 mmol) and EDC×HCl (0.53 g, 2.8 mmol) were added and the reaction mixture was stirred for additional 4 h. The solvent was removed under reduced pressure followed by addition of water (10 mL) and extraction with EtOAc (3×10 mL). The combined organics were dried (Na₂SO₄), filtered and concentrated. The afforded crude was used in the next step without further purification. MS m/z 277 [M+1]⁺.

### Step b: N-(3-(2-cyclohexylethyl)-2,4-dioxo-1,2,3,4-tetrahydroquinazolin-6-yl)pivalamide (15b)

The crude compound from the previous step was reacted with cyclohexylethylamine followed by carbonylation and ring closure according to the method described in Example 14 steps b and c, which gave the title compound (0.075 g, 20%).

¹H NMR (DMSO-d₆, 400 MHz): δ 11.35 (1H, s), 9.97 (1H, s), 8.24 (1H, d, J=2.4 Hz), 7.79 (1H, dd, J=8.8, 2.4 Hz), 7.11 (1H, d, J=8.8 Hz), 3.92-3.88 (2H, m), 2.18 (2H, s), 1.80-1.70 (2H, m), 1.70-1.60 (3H, m), 1.47-1.43 (2H, m), 1.25-1.22 (4H, m), 1.0 (9H, m), 0.90-0.80 (2H, m). MS: m/z 385 [M−1]⁻.

### Example 16

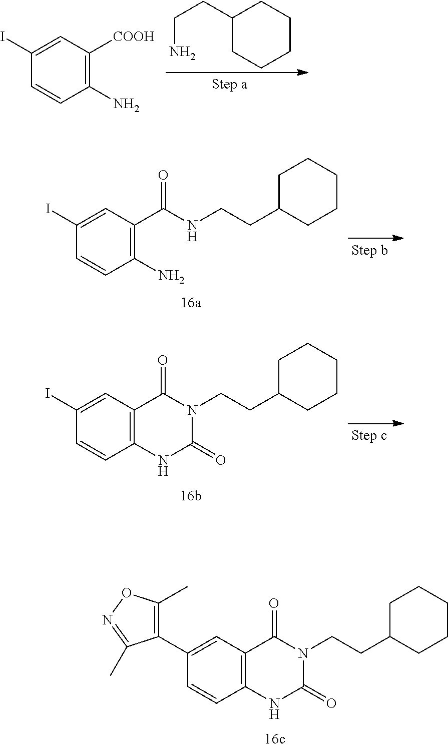 [SMILES: Nc1ccc(I)cc1C(=O)O ; NCCC1CCCCC1 ; Nc1ccc(I)cc1C(=O)NCCC1CCCCC1 ; O=c1[nH]c2ccc(I)cc2c(=O)n1CCC1CCCCC1 ; Cc1noc(C)c1-c1ccc2[nH]c(=O)n(CCC3CCCCC3)c(=O)c2c1]

### Step a) 2-amino-N-(2-cyclohexylethyl)-5-iodobenzamide (16a)

HATU (6.84 g, 18 mmol) was added to a cold (0° C.) solution of 2-amino-5-iodo benzoic acid (4 g, 15 mmol) in DMF (80 mL). The solution was stirred for 5 min, then cyclohexyl ethylamine (1.93 g, 15 mmol) and NMM (4.5 g, 45 mmol) were added. The reaction mixture was stirred at room temperature for 16 h, concentrated under reduced pressure. Water (50 mL) was added and the mixture was extracted with EtOAc (3×50 mL). The combined organics were dried (Na₂SO₄), filtered and concentrated. The afforded crude was taken to the next step without further purification. MS: m/z 373 [M+1]⁺.

### Step b) 3-(2-cyclohexylethyl)-6-iodoquinazoline-2,4(1H,3H)-dione (16b)

The crude material from previous step (2 g) was taken in ethylchloroformate (7.6 mL) and heated to 90° C. for 1.5 h. The solvent was removed under reduced pressure and the crude was dissolved in EtOH (60 mL) followed by addition of KOH (0.45 g, 8.06 mmol) and heating at 85° C. for 3 h. The solvent was removed under reduced pressure followed by addition of water (100 mL). The solution was acidified with glacial acetic acid till pH 7 and extracted with EtOAc (3×50 mL). The combined organics were dried (Na₂SO₄), filtered and concentrated. The obtained solid (2 g, 33% after 2 steps) was used in the next step. MS: m/z 397 (M⁺−1).

### Step c) 3-(2-Cyclohexylethyl)-6-(3,5-dimethylisoxazol-4-yl)quinazoline-2,4(1H,3H)-dione (16c)

Water (1 mL), Na₂CO₃ (0.16 g, 1.5 mmol) and 3,5-dimethylisoxazol-4-yl-boronic-acid (0.14 g, 1 mmol) were added to a solution of iodo derivative 16b (0.2 g, 0.50 mmol) in 1,4-dioxane (2 mL). The solution was degasified, then, Pd (Ph₃P)₄ (0.12 g, 0.01 mmol) was added under nitrogen. The reaction mixture was heated at 90° C. for 16 h, then water (20 mL) was added and the mixture was extracted with EtOAc (3×20 mL). The combined organic phases were dried (Na₂SO₄), filtered and concentrated. The crude was purified by flash column chromatography on silica gel which gave the title compound (0.03 g, yield 32% based on recovered starting material) as a solid.

¹H NMR (DMSO-d₆, 400 MHz): δ 11.53 (1H, s), 7.85 (1H, d, J=2 Hz), 7.68 (1H, dd, J=8.4, 2 Hz), 7.27 (1H, d, J=8.4 Hz), 3.94-3.90 (2H, m), 2.49 (3H, s), 2.39 (3H, s), 1.80-1.70 (2H, m), 1.70-1.59 (3H, m), 1.55-1.40 (2H, m), 1.29-1.11 (4H, m), 0.90-0.70 (2H, m). MS: m/z 366 [M−1]⁻.

### Example 17

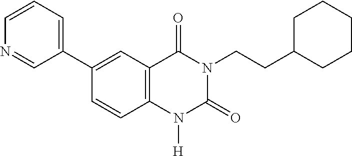 [SMILES: O=c1[nH]c2ccc(-c3cccnc3)cc2c(=O)n1CCC1CCCCC1]

### 3-(2-Cyclohexylethyl)-6-(pyridin-3-yl)quinazoline-2,4(1H,3H)-dione (17)

Pyridine-3-boronic acid (0.04 g, 0.37 mmol) was reacted with the iodo derivative 16b (0.1 g, 0.25 mmol) according to the method described in Example 16 step c, but using Cs₂CO₃ instead of Na₂CO₃, which gave the title compound (0.05 g, 57%).

¹H NMR (DMSO-d₆, 400 MHz): δ 11.56 (1H, s), 8.90 (1H, d, J=2 Hz), 8.57 (1H, dd, J=8, 2 Hz), 8.19 (1H, d, J=2 Hz), 8.10 (1H, d, J=8 Hz), 8.05 (1H, dd, J=8.4, 2.4 Hz), 7.62-7.48 (1H, m), 7.29 (1H, d, J=8.4 Hz), 3.95-3.91 (2H, m), 1.77-1.74 (2H, m), 1.67-1.59 (3H, m), 1.49-1.44 (2H, m), 1.28-1.22 (1H, m), 1.19-1.11 (3H, m), 0.97-0.90 (2H, m). MS: m/z 348 [M−1]⁻.

### Example 18

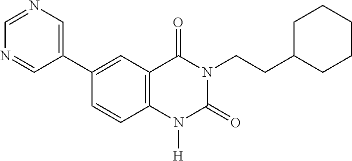 [SMILES: O=c1[nH]c2ccc(-c3cncnc3)cc2c(=O)n1CCC1CCCCC1]

### 3-(2-Cyclohexylethyl)-6-(pyrimidin-5-yl)quinazoline-2,4(1H,3H)-dione (18)

Pyrimidine-5-boronic acid (0.045 g, 0.37 mmol) was reacted with the iodo derivative 16b (0.1 g, 0.25 mmol) according to the method described in Example 16 step c, but using Cs₂CO₃ instead of Na₂CO₃, which gave the title compound (0.035 g, 40%).

¹H NMR (DMSO-d₆, 400 MHz): δ 11.60 (1H, s), 9.18 (1H, s), 9.16 (2H, s), 8.29 (1H, d, J=2 Hz), 8.10 (1H, dd, J=8.4, 2 Hz), 7.31 (1H, d, J=8.4 Hz), 3.95-3.91 (2H, m), 1.77-1.73 (2H, m), 1.65-1.59 (3H, m), 1.49-1.44 (2H, m), 1.33-1.11 (4H, m), 0.97-0.88 (2H, m). MS: m/z 349 [M−1]⁻.

### Example 19

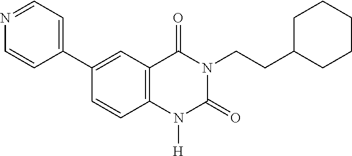 [SMILES: O=c1[nH]c2ccc(-c3ccncc3)cc2c(=O)n1CCC1CCCCC1]

### 3-(2-Cyclohexylethyl)-6-(pyridin-4-yl)quinazoline-2,4(1H,3H)-dione (19)

Pyridine-4-boronic acid (0.045 g, 0.37 mmol) was reacted with the iodo derivative 16b (0.1 g, 0.25 mmol) according to the method described in Example 16 step c, but using Cs₂CO₃ instead of Na₂CO₃, which gave the title compound (0.055 g, 63%).

¹H NMR (DMSO-d₆, 400 MHz): δ 11.61 (1H, s), 8.64 (2H, d, J=5.2 Hz), 8.28 (1H, d, J=2 Hz), 8.12 (1H, dd, J=8.4, 2 Hz), 7.73 (2H, d, J=5.2 Hz), 7.30 (1H, d, J=8.4 Hz), 3.95-3.91 (2H, m), 1.77-1.74 (2H, m), 1.67-1.59 (3H, m), 1.49-1.44 (2H, m), 1.28-1.22 (1H, m), 1.19-1.11 (3H, m), 0.97-0.90 (2H, m). MS: m/z 348 [M−1]⁻.

### Example 20

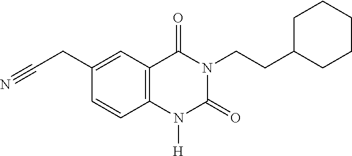 [SMILES: N#CCc1ccc2[nH]c(=O)n(CCC3CCCCC3)c(=O)c2c1]

### 2-(3-(2-cyclohexylethyl)-2,4-dioxo-1,2,3,4-tetrahydroquinazolin-6-yl)acetonitrile (20)

Pd(Ph₃P)₄ (0.06 g, 0.005 mmol) was added under nitrogen to a degassed solution of iodo derivative 16b (0.1 g, 0.25 mmol) in 1,4-dioxane (1 mL), water (0.5 mL), Na₂CO₃ (0.08 g, 0.7 mmol) and isoxazol-4-yl-boronic-acid (0.04 g, 0.4 mmol) in a screw capped reaction vessel.

The vessel was tightly sealed and the reaction mixture was heated at 90° C. for 16 h. The vessel was opened at room temperature, then water (10 mL) was added and the mixture was extracted with EtOAc (3×10 mL). The combined organics was dried (Na₂SO₄), filtered and concentrated. The crude was purified by flash column chromatography on silica gel, which gave the title compound (0.03 g, yield 42%) as a solid.

¹H NMR (DMSO-d₆, 400 MHz): δ 11.46 (1H, s), 7.91 (1H, s), 7.60 (1H, m), 7.18 (1H, d, J=8.8 Hz), 4.08 (2H, s), 3.95-3.88 (2H, m), 1.75-1.73 (2H, m), 1.67-1.59 (3H, m), 1.47-1.44 (2H, m), 1.27-1.11 (4H, m), 0.97-0.90 (2H, m). MS: m/z 310 (M⁺−1).

### Example 21

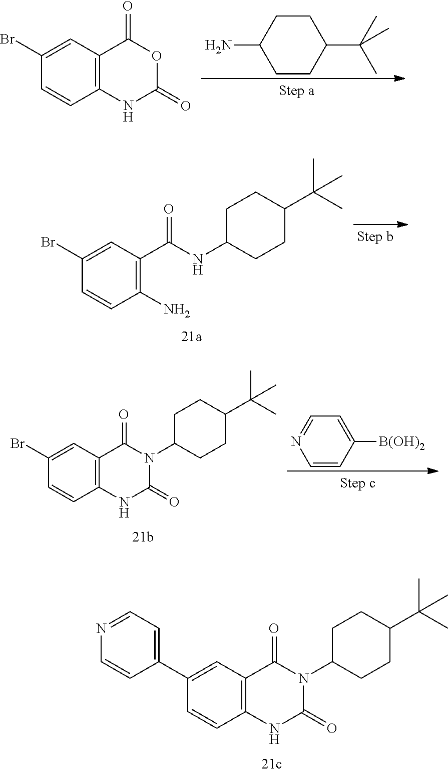

### Step a) 2-amino-5-bromo-N-(4-(tert-butyl)cyclohexyl)benzamide (21a)

DMAP (20 mg) and 5-bromo isatoic anhydride (0.5 g, 2.05 mmol) were added to a cold (0° C.) solution of 4-tert-butyl-cyclohexyl-amine (0.48 g, 3.08 mmol) in DMF (4 mL). The solution was stirred at room temperature for 3 h, then the solvent was removed under reduced pressure, water (20 mL) was added and the mixture was extracted with EtOAc (3×20 mL). The combined organics were dried (Na₂SO₄), filtered and concentrated. The crude was taken to the next step without further purification.

MS: m/z 353 [M+1]⁺.

### Step b) 6-bromo-3-(4-(tert-butyl)cyclohexyl)quinazoline-2,4(1H,3H)-dione (21b)

The crude material from step a (0.5 g), was taken in ethylchloroformate (2 mL) and heated to 90° C. for 1.5 h. The solvent was removed under reduced pressure and the crude was dissolved in EtOH (12 mL) followed by addition of KOH (0.17 g, 3.1 mmol) and heating at 85° C. for 3 h. The solvent was removed under reduced pressure, water (10 mL) was added and the mixture was extracted with EtOAc (3×10 mL). The combined organics were dried (Na₂SO₄), filtered and concentrated. The crude was purified by flash column chromatography on silica gel, which gave the title compound (0.4 g, 51% after 2 steps) as a solid.

MS: m/z 378 [M+1]⁺.

### Step c) 3-(4-(tert-butyl)cyclohexyl)-6-(pyridin-4-yl)quinazoline-2,4(1H,3H)-dione (21c)

Water (1 mL), Cs₂CO₃ (0.55 g, 1.5 mmol) and 4-pyridine-boronic-acid (0.11 g, 0.79 mmol) were added to a solution of bromo derivative 21b (0.2 g, 0.52 mmol) in 1,4-dioxane (2 mL). The solution was degassed, then Pd (PPh₃)₄(0.13 g, 0.021 mmol) was added under nitrogen. The reaction mixture was heated at 70° C. for 16 h, then water (20 mL) was added and the mixture was extracted with EtOAc (3×20 mL). The combined organics were dried (Na₂SO₄), filtered and concentrated. The crude was purified by flash column chromatography on silica gel which gave the title compound (0.11 g, yield 55%).

¹H NMR (DMSO-d₆, 400 MHz): δ 11.52 (1H, s), 8.64-8.63 (2H, m,), 8.28-8.26 (1H, m), 8.11-8.08 (1H, m), 7.74-7.73 (2H, m), 7.63-7.56 (1H, m), 7.28-7.25 (1H, m), 4.74-4.69 (1H, m), 2.52-2.37 (2H, m), 1.85-1.83 (2H, m), 1.67-1.65 (2H, m), 1.60-1.45 (1H, m), 1.22-1.06 (2H, m), 0.91 & 0.86 (9H, s each). MS: m/z 378 (M⁺+1).

### Example 22

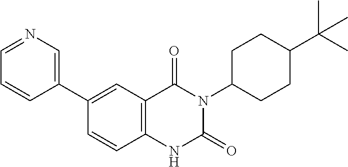 [SMILES: CC(C)(C)C1CCC(n2c(=O)[nH]c3ccc(-c4cccnc4)cc3c2=O)CC1]

### 3-(4-(tert-butyl)cyclohexyl)-6-(pyridin-3-yl)quinazoline-2,4(1H,3H)-dione (22)

Bromo derivative 21b (0.2 g, 0.52 mmol) was reacted with 3-pyridine-boronic-acid (0.11 g, 0.79 mmol) according to the procedure described in Example 22 step c, which gave the title compound (0.10 g, 50%).

¹H NMR (DMSO-d₆, 400 MHz): δ 11.46 (1H, s), 8.89 (1H, br s), 8.57 (1H, br s), 8.18-8.16 (1H, m), 8.10-8.08 (1H, m), 8.03-8.01 (1H, m), 7.50-7.47 (1H, m), 7.27-7.24 (1H, m), 4.75-4.69 (1H, m), 2.50-2.37 (2H, m), 1.85-1.83 (2H, m), 1.67-1.65 (2H, m), 1.60-1.45 (1H, m), 1.22-1.06 (2H, m), 0.91 & 0.86 (9H, s each). MS: m/z 376 [M−1]⁻.

### Example 23

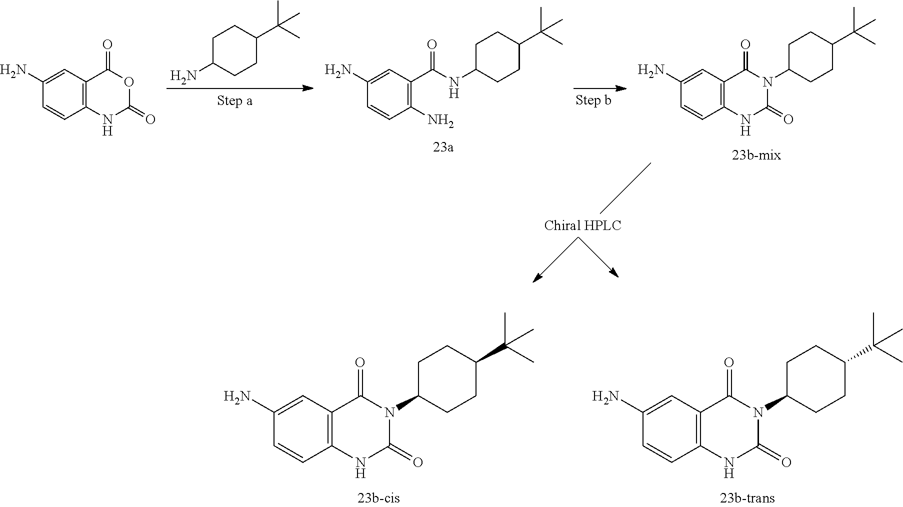 [SMILES: Nc1ccc2[nH]c(=O)oc(=O)c2c1 ; CC(C)(C)C1CCC(N)CC1 ; CC(C)(C)C1CCC(NC(=O)c2cc(N)ccc2N)CC1 ; CC(C)(C)C1CCC(n2c(=O)[nH]c3ccc(N)cc3c2=O)CC1 ; CC(C)(C)[C@H]1CC[C@@H](n2c(=O)[nH]c3ccc(N)cc3c2=O)CC1 ; CC(C)(C)[C@H]1CC[C@H](n2c(=O)[nH]c3ccc(N)cc3c2=O)CC1]

### Step a) 2,5-diamino-N-(4-tert-butyl-cyclohexyl)-benzamide (23a)

DMAP (0.04 g, 0.3 mmol) was added to a cooled (0° C.) solution of a cis-& trans-mix of 4-tert-butylcyclohexyl amine (1.2 g, 8.5 mmol) in DMF (10 mL), followed by addition of 5-aminoisatoic anhydride (1 g, 5.6 mmol). The solution was stirred for 3 h at room temperature, then concentrated under reduced pressure. Water (10 mL) was added and the mixture was and extracted with EtOAc (3×20 mL). The combined organics were concentrated under reduced pressure and the afforded crude was taken to the next step without further purification. MS: m/z 288 [M+1]⁻.

### Step b) 6-amino-3-(4-tert-butyl-cyclohexyl)-1H-quinazoline-2,4-dione (23b-mix)

The crude amine 23a (0.9 g, 3.13 mmol) and ethylchloroformate (3.4 mL) was heated at 90° C. for 1.5 h. The solvent was removed under reduced pressure and the crude was dissolved in EtOH (33 mL). KOH (0.36 g, 6.26 mmol) was added and the mixture was heated at 85° C. overnight. The solvent was removed under reduced pressure, water (10 mL) was added and the mixture was extracted with EtOAc (3×20 mL). The combined organics were dried (Na₂SO₄), filtered and concentrated. The afforded crude was purified by flash column chromatography on silica gel which gave the title compound as a mixture of cis and trans isomers (0.3 g, 30%).

¹H NMR (DMSO-d₆, 400 MHz): δ 10.90+10.88 (1H, s each), 7.08 (1H, d, J=2 Hz), 6.92-6.85 (2H, m), 5.15 (2H, brs), 4.91+4.69 (1H, m each), 2.55-2.32 (2H, m), 1.84-1.81 (1H, m), 1.71-1.68 (1H, m), 1.61-1.58 (1H, m), 1.49-1.42 (2H, m), 1.35-1.2 (1H, m), 1.14-1.05 (1H, m), 0.90+0.86 (9H, s each). MS: m/z 316 [M+1]⁺.

### Separation of Diastereomers

The two diastereomers were separated by chiral prep. HPLC using a CHIRAL Phenomenox Lux Cellulose-4 (250×4.6) mm, 5 μm; Flow: 1.0 mL/min; Mobile phase A: Hexanes:EtOH (70:30);

### 6-amino-3-(cis-4-tert-butyl-cyclohexyl)-1H-quinazoline-2,4-dione (23b-cis)

Chiral HPLC: Retention time: 8.19 min.

¹H NMR (DMSO-d₆, 400 MHz): δ 10.88 (1H, s), 7.08 (1H, s), 6.93-6.85 (2H, m), 5.14 (2H, brs), 4.92-4.85 (1H, m), 2.40-2.25 (2H, m), 1.75-1.65 (2H, m), 1.55-1.40 (4H, m), 1.35-1.25 (1H, m), 0.90 (9H, s).

### 6-amino-3-(trans-4-tert-butyl-cyclohexyl)-1H-quinazoline-2,4-dione (23b-trans)

Chiral HPLC: Retention time: 10.41 min.

¹H NMR (DMSO-d₆, 400 MHz): δ 10.90 (1H, s), 7.08 (1H, s), 6.93-6.85 (2H, m), 5.17 (2H, brs), 4.73-4.65 (1H, m), 2.50-2.40 (2H, m), 1.84-1.80 (2H, m), 1.62-1.58 (2H, m), 1.10-1.04 (3H, m), 0.86 (9H, s).

### Alternative route for the synthesis of 6-amino-3-(trans-4-tert-butyl-cyclohexyl)-1H-quinazoline-2,4-dione

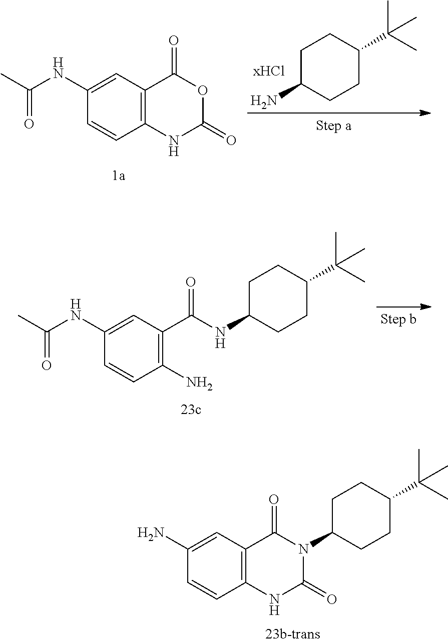 [SMILES: CC(=O)Nc1ccc2[nH]c(=O)oc(=O)c2c1 ; CC(C)(C)[C@H]1CC[C@H](N)CC1 ; CC(=O)Nc1ccc(N)c(C(=O)N[C@H]2CC[C@H](C(C)(C)C)CC2)c1 ; CC(C)(C)[C@H]1CC[C@H](n2c(=O)[nH]c3ccc(N)cc3c2=O)CC1]

### Step a) 5-Acetylamino-2-amino-N-(trans-4-tert-butyl-cyclohexyl)-benzamide (23c)

Et₃N (3.57 mL, 26 mmol) and isatoic anhydride derivative 1a (5 g, 22 mmol) were added to a solution of trans-4-tert-butyl-cyclohexylaminexhydrochloride (6.5 g, 34 mmol) in DMF (40 mL) at 0° C. The solution was stirred at room temperature for 3 h, then concentrated under reduced pressure followed by addition of water and extraction with EtOAc (3×100 mL). The combined organics were washed with brine, dried (Na₂SO₄), filtered and concentrated. The afforded crude was purified by flash column chromatography on silica gel, which gave the title compound (3.6 g, 49%).

¹H NMR (DMSO-d₆, 400 MHz): δ 9.57 (1H, s), 7.93 (1H, d, J=2.4 Hz), 7.39 (1H, d, J=2.4 Hz), 7.33 (1H, dd, J=8.4, 2.4 Hz), 6.60 (1H, d, J=8.4 Hz), 5.91 (2H, s), 3.64-3.60 (1H, m), 1.95 93H, s), 1.86-1.84 (2H, m), 1.76-1.73 (2H, m), 1.32-1.21 (2H, m), 1.09-0.9 (3H, m), 0.84 (9H, s).

MS: m/z 332 (M⁺+1).

### Step b) 6-amino-3-(trans-4-tert-butyl-cyclohexyl)-1H-quinazoline-2,4-dione (23b-trans)

Compound 23c (3.2 g, 9.6 mmol) was taken in ethylchloroformate (11.84 mL) and heated to 90° C. for 1.5 h. The solvent was removed under reduced pressure and the crude was dissolved in EtOH (64 mL) followed by addition of KOH (4.4 g, 77 mmol) and heating at 85° C. for 16 h. The solvent was removed under reduced pressure followed by addition of water (100 mL) and extraction with EtOAc (3×100 mL). The combined organics was dried (Na₂SO₄), filtered and concentrated. The crude was purified by flash column chromatography on silica gel, which gave the title compound (1.4 g, 46%) as a brown coloured solid.

### Example 24

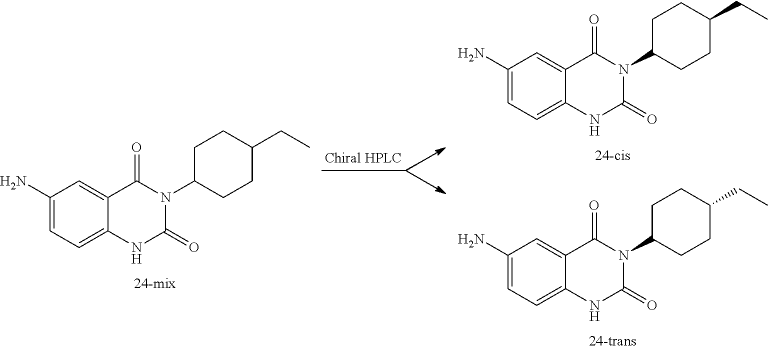

### 6-Amino-3-(4-ethyl-cyclohexyl)-1H-quinazoline-2,4-dione, cis trans mixture (24-mix)

The title compound was prepared from 5-aminoisatoic anhydride (1 g, 5.6 mmol) and 4-ethylcyclohexyl amine (cis, trans mix, 1.17 g, 8.5 mmol) according to the procedure described in Example 23 steps a and b. Yield: 0.4 g, 51%.

¹H NMR (DMSO-d₆, 400 MHz): δ 10.89&10.88 (1H, s each), 7.08 (1H, d, J=2 Hz), 6.93-6.85 (2H, m), 5.15 (2H, s), 4.75-4.68 (1H, m), 2.45-2.39 (2H, m), 1.83-1.80 & 1.70-1.67 (2H, m each), 1.56-1.46 (3H, m), 1.41-1.31 (2H, m), 1.31-1.28 & 1.20 & 1.14 (3H, m each), 1.02-0.95 (1H, m), 0.90-0.88 (3H, m). MS: m/z 288 [M+1]⁺.

### Separation of Diastereomers

The two diastereomers were separated by chiral prep. HPLC using a CHIRAL Phenomenox Lux Cellulose-4 (250×4.6) mm, 5 μm; Flow: 1.0 mL/min; Mobile phase A: Hexanes:EtOH (70:30).

### 6-Amino-3-(cis-4-ethyl-cyclohexyl)-1H-quinazoline-2,4-dione (24-cis)

Chiral HPLC: Retention time: 7.32 min.

¹H NMR (DMSO-d₆, 400 MHz): δ 10.87 (1H, s), 7.07 (1H, d, J=2 Hz), 6.94-6.85 (2H, m), 5.15 (2H, s), 4.76-4.69 (1H, m), 2.60-2.50 (2H, m), 1.71-1.67 (2H, m), 1.53-1.45 (5H, m), 1.32-1.25 (2H, m), 0.88 (3H, t, J=7 Hz).

### 6-Amino-3-(trans-4-ethyl-cyclohexyl)-1H-quinazoline-2,4-dione (24-trans)

Chiral HPLC: Retention time: 8.32 min.

¹H NMR (MeOD, 400 MHz): δ 7.30 (1H, d, J=2.8 Hz), 7.07 (1H, dd, J=8.8, 2.8 Hz), 6.94 (1H, d, J=8.8 Hz), 4.85-4.81 (1H, m), 2.60-2.50 (2H, m), 1.93-1.90 (2H, m), 1.67-1.64 (2H, m), 1.32-1.26 (3H, m), 1.12-1.04 (2H, m), 0.94 (3H, t, J=8 Hz).

### Example 25

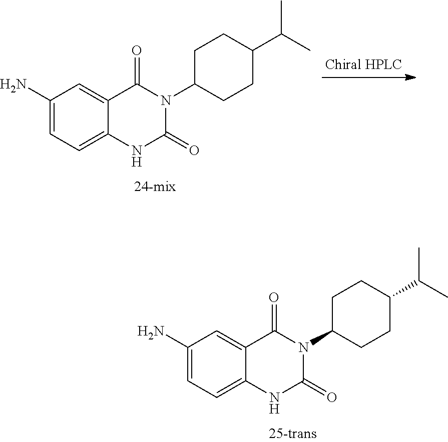 [SMILES: CC(C)C1CCC(n2c(=O)[nH]c3ccc(N)cc3c2=O)CC1 ; CC(C)[C@H]1CC[C@H](n2c(=O)[nH]c3ccc(N)cc3c2=O)CC1]

### 6-Amino-3-(4-isopropylcyclohexyl)-1H-quinazoline-2,4-dione (25-mix)

The title compound was prepared from 5-aminoisatoic anhydride (1 g, 5.6 mmol) and 4-isopropylcyclohexyl amine (cis, trans mix, 1.18 g, 8.5 mmol) according to the procedure described in Example 23 steps a and b. Yield: 0.4 g, 53%

¹H NMR (DMSO-d₆, 400 MHz): δ 10.89 & 10.86 (1H, s each), 7.08 (1H s), 6.93-6.85 (2H m), 5.15 (2H, s), 4.76-4.69 (1H, m), 2.50-2.41 (2H, m), 2.0-1.60 (2H, m), 1.59-1.56 (2H, m), 1.50-1.25 (1H, m), 1.07-1.03 (3H, m), 0.90 & 0.86 (6H. d each, J=6.8 Hz). MS: m/z 302 (M⁺+1).

### 6-Amino-3-((1 r,4r)-4-isopropylcyclohexyl)quinazoline-2,4(1H,3H)-dione (25-trans)

The trans isomer was isolated by chiral prep HPLC using a CHIRAL PAK IC (250×4.6) mm, 5μ; Flow: 1.0 mL/min; Mobile phase A: Hexanes:IPA (90:10), retention time: 13.72 min.

¹H NMR (DMSO-d₆, 400 MHz): δ 10.89 (1H, s), 7.08 (1H, d, J=2.4 Hz), 6.93-6.8 (2H, m), 5.15 (2H, m), 4.73-4.65 (1H, m), 2.50-2.40 (2H, m), 1.77-1.75 (2H, m), 1.59-1.56 (2H, m), 1.45 (1H, brm), 1.07-1.03 (3H, m), 0.86 (6H, d, J=6.8). MS: m/z 302 (M+1)⁺.

### Example 26

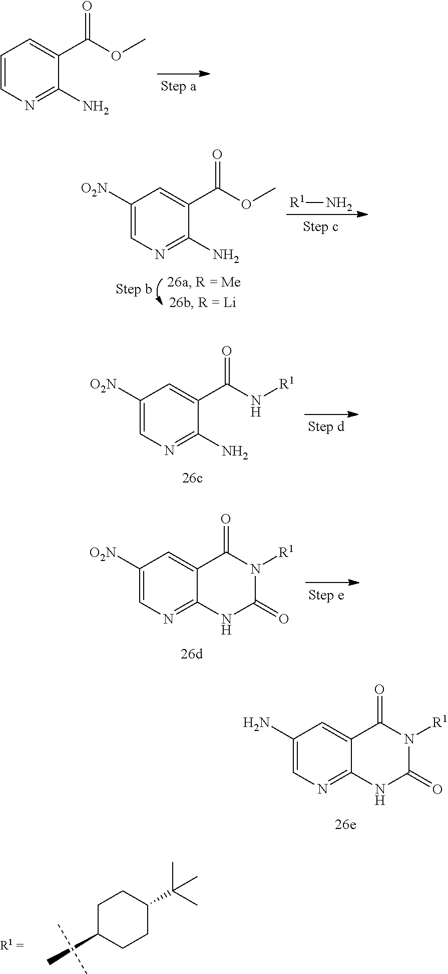

### Step a) 2-Amino-5-nitronicotinic acid methyl ester (26a)

A solution of 2-amino-3-nicotinic acid methyl ester (4 g, 26 mmol) in a mixture of concentrated HNO₃ (2.8 mL) and H₂SO₄ (10 mL) was stirred for 45 min at 0° C., followed by room temperature for 19 h, and at 70° C. for 4 h. The reaction mixture was cooled to 0° C. and a saturated aqueous solution of NaHCO₃ (40 mL) was added till basic (pH 8). Extraction with EtOAc (3×40 mL), filtered and concentration of the combined organics afforded the title compound (3.5 g, 68%) which was used in the next step without further purification.

¹H NMR (DMSO-d₆, 400 MHz): δ 9.05 (1H, d, J=2.8 Hz), 8.69 (1H, d, J=2.8 Hz), 8.64 (1H brs), 8.15 (1H, brs), 3.88 (3H, s). MS: m/z 198 (M+1)⁺.

### Step b) Lithium salt of 2-amino-5-nitronicotinic acid (26b)

LiOH (0.12 g, 5 mmol) was added to a solution of the methyl ester 26a (1 g, 5 mmol) in a mixture of 1% MeOH in THF (10 mL). The solution was stirred at room temperature for 17 h, then concentrated under reduced pressure. The solid obtained (0.8 g) was used in the next step without further purification.

### Step c) 2-Amino-N-(trans-4-tert-butyl-cyclohexyl)-5-nitro-nicotinamide (26c)

BOP (2.7 g, 6 mmol) was added to a suspension of the lithium salt 26b (0.8 g, 4 mmol), trans-4-^{t}Butylcyclohexylamine hydrochloride salt (1.16 g, 6 mmol) and triethylamine (1.2 g, 12 mmol) in DMF. The suspension was stirred at room temperature for 6 h, then concentrated under reduced pressure. Water (15 mL) was added and the mixture was extracted with EtOAc (3×20 mL). The combined organics was dried (Na₂SO₄), filtered and concentrated under reduced pressure. The crude obtained was purified by flash column chromatography on silica gel, which gave the title compound (0.8 g, 62%).

¹H NMR (DMSO-d₆, 400 MHz): δ 9.57 (1H, s), 7.93 (1H, d, J=8 Hz), 7.38 (1H d, J=2.4), 5.91 (2H, s), 3.65-3.60 (1H, m), 1.86-1.84 (2H, m), 1.77-1.73 (2H, m), 1.35-1.20 (2H, m), 1.09-0.95 (3H, m), 0.84 (9H, s). MS: m/z 321 (M⁺+1)

### Step d) 3-(trans-4-tert-Butyl-cyclohexyl)-6-nitro-1H-pyrido[2,3-d]pyrimidine-2,4-dione (26d)

1,1′-Carbonyldiimidazole (1.06 g, 6.5 mmol) was added to a solution of compound 26c (0.7 g, 2.1 mmol) in THF (7 mL), and the solution was heated at 90° C. for 48 h. The solvent was removed under reduced pressure, water (10 mL) was added and the mixture was extracted with EtOAc (3×20 mL). The combined organic phases were concentrated under reduced pressure and the afforded crude was purified by flash column chromatography on silica gel which gave the title compound (0.6 g, 82%).

¹H NMR (DMSO-d₆, 400 MHz): δ 10.89 (1H, s), 7.07 (1H, d, J=2.4 Hz), 6.89 (1H, d, J=2.4 Hz), 4.72-4.65 (1H, m), 2.50-2.38 (2H, m), 1.83-1.80 (2H, m), 1.60-1.57 (2H, m), 1.08-1.04 (3H, m), 0.85 (9H, m). MS: m/z 345 (M−1)⁻.

### 6-Amino-3-(trans-4-tert-butyl-cyclohexyl)-1H-pyrido[2,3-d]pyrimidine-2,4-dione (26e)

A solution of the nitro compound 26d (0.6 g, 1.7 mmol) in 30% CH₂Cl₂ in MeOH (9 mL) was purged with N₂ (g) followed by addition of 10% Pd in C (0.12 g). The reaction was hydrogenated at atmospheric pressure for 3 h, then filtered through a Celite bed and the filtrate was concentrated under reduced pressure. The crude was purified by flash column chromatography on silica gel which gave the title compound (0.11 g, 21%).

¹H NMR (DMSO-d₆, 400 MHz): δ 11.36 (1H, s), 8.00 (1H, s), 7.44 (1H, s), 5.36 (2H, s), 4.68-4.62 (1H, m), 2.42-2.32 (2H, m), 1.83-1.81 (2H, m), 1.62-1.59 (2H, m), 1.10-1.04 (3H, m), 0.86 (9H, s). MS: m/z 317 (M⁺+1)

The following compounds were prepared from the lithium salt 26b according to the procedure described in Example 26 steps a-e, using the indicated amine R¹—NH₂:

| | | | | MS |
|---|---|---|---|---|
| Ex | Structure | R¹—NH₂ | Yield | [M + 1]⁺ |
| 27 | 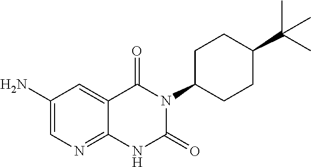 [SMILES: CC(C)(C)[C@H]1CC[C@@H](n2c(=O)[nH]c3ncc(N)cc3c2=O)CC1] | (1S,4S)-4-(Tert- butyl)- cyclohexanamine | 11% | 317 |
| | 6-Amino-3-(( 1 S,4S)-4-(tert-butyl)- | | | |
| | cyclohexyl)pyrido[2,3-d]pyrimidine-2,4(1H.3H)- | | | |
| | dione (27) | | | |
| | ¹H NMR (DMSO-d₆, 400 MHz): δ 11.37 (1H, s), 8.01 (1H, d, J = 2.8 Hz), 7.44 (1H, d, J = | | | |
| | 2.8 Hz), 5.37 (2H, s), 4.92-4.84 (1H, m), 2.33-2.30 (2H, m), 1.72-1.68 (4H, m), 1.32- | | | |
| | 1.31 (1H, m), 1.33-1.30 (1H, m), 0.90 (9H, s). | | | |
| 28 | 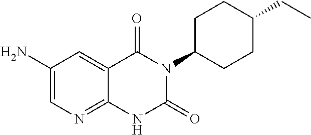 [SMILES: CC[C@H]1CC[C@H](n2c(=O)[nH]c3ncc(N)cc3c2=O)CC1] | (1R,4R)-4- Ethylcyclohexan- amine | 24% | 289 |
| | 6-Amino-3-((1R,4R)-4-ethylcyclohexyl)pyrido[2,3- | | | |
| | d]pyrimidine-2,4(1H,3H)-dione (28) | | | |
| | ¹H NMR (DMSO-d₆, 400 MHz): δ 11.38 (1H, s), 8.017 (1H, d, J = 2.8 Hz), 7.44 (1H, d, J = | | | |
| | 2.8 Hz), 5.37 (2H, s), 4.72-4.65 (1H, m), 2.41-2.37 (2H, m), 1.83-1.80 (2H, m), 1.58- | | | |
| | 1.55 (2H, m), 1.25-1.10 (3H, m), 1.0-0.9 (2H, m), 0.87 (3H, t, J = 7.2 Hz). | | | |
| 29 | 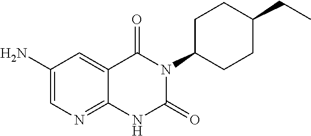 [SMILES: CC[C@H]1CC[C@@H](n2c(=O)[nH]c3ncc(N)cc3c2=O)CC1] | (1S,4S)-4- Ethylcyclohexan- amine | 26% | 289 |
| | 6-Amino-3-((1S,4S)-4-ethylcyclohexyl)- | | | |
| | pyrido[2,3-d]pyrimidine-2,4(1H,3H)-dione (29) | | | |
| | ¹H NMR (DMSO-d₆, 400 MHz): δ 11.38 (1H, s), 8.017 (1H, d, J = 2.8 Hz), 7.44 (1H, d, J = | | | |
| | 2.8 Hz), 5.37 (2H, s), 4.72-4.65 (1H, m), 2.41-2.37 (2H, m), 1.83-1.80 (2H, m), 1.58- | | | |
| | 1.55 (2H, m), 1.25-1.10 (3H, m), 1.0-0.9 (2H, m), 0.87 (3H, t, J = 7.2 Hz). | | | |
| 30 | 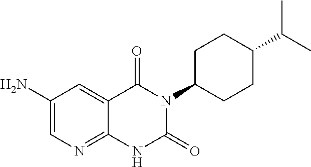 [SMILES: CC(C)[C@H]1CC[C@H](n2c(=O)[nH]c3ncc(N)cc3c2=O)CC1] | (1R,4R)-4-Isopropyl- cyclohexanamine | 11% | 303 |
| | 6-Amino-3-((1R,4R)-4-isopropyl- | | | |
| | cyclohexyl)pyrido[2,3-d]pyrimidine-2,4(1H,3H)- | | | |
| | dione (30) | | | |
| | ¹H NMR (DMSO-d₆, 400 MHz): δ 11.37 (1H, s), 8.01 (1H, d, J = 2.8 Hz), 7.44 (1H, d, | | | |
| | J = 2.8 Hz), 5.37 (2H, s), 4.70-4.63 (1H, m), 2.45-2.34 (2H, m), 1.78-1.74 (2H, m), 1.62- | | | |
| | 1.50 (2H, m), 1.45-1.43 (1H, m), 1.08-1.04 (3H, m), 0.86 (6H, d, J = 8 Hz). | | | |
| 31 | 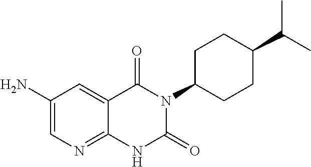 [SMILES: CC(C)[C@H]1CC[C@@H](n2c(=O)[nH]c3ncc(N)cc3c2=O)CC1] | (1S,4S)-4-Isopropyl- cyclohexanamine | 12% | 303 |
| | 6-Amino-3-((1S,4S)-4-isopropyl- | | | |
| | cyclohexyl)pyrido[2,3-d]pyrimidine-2,4(1H,3H)- | | | |
| | dione (31) | | | |
| | ¹H NMR (DMSO-d₆, 400 MHz): δ 11.37 (1H, s) 8.01 (1H, d, J = 2.8 Hz), 7.42 (1H, d, | | | |
| | J = 2.8 Hz), 5.37 (2H, s), 4.75-4.69 (1H, m), 2.49-2.40 (2H, m), 1.97-1.85 (3H, m), 1.42- | | | |
| | 1.30 (4H, m), 1.15-1.13 (1H, m), 0.86 (6H, d, J = 8 Hz). | | | |
| 32 | 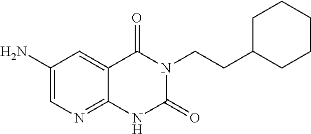 [SMILES: Nc1cnc2[nH]c(=O)n(CCC3CCCCC3)c(=O)c2c1] | 2- Cyclohexylethanamine | na | na |
| | 6-Amino-3-(2-cyclohexylethyl)-pyrido[2,3- | | | |
| | d]pyrimidine-2,4(1H,3H)-dione (32) | | | |

### Example 33

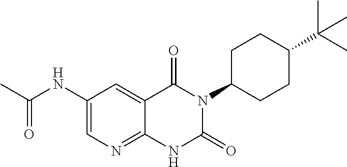 [SMILES: CC(=O)Nc1cnc2[nH]c(=O)n([C@H]3CC[C@H](C(C)(C)C)CC3)c(=O)c2c1]

### N-[3-(trans-4-tert-Butyl-cyclohexyl)-2,4-dioxo-1,2,3,4-tetrahydro-pyrido[2,3-d]pyrimidin-6-yl]-acetamide (33)

Triethylamine (0.076 g, 0.75 mmol) was added at 0° C. to a solution of 6-amino-3-(trans-4-tert-butyl-cyclohexyl)-1H-pyrido[2,3-d]pyrimidine-2,4-dione (0.08 g, 0.25 mmol) in CH₂Cl₂ (2 mL). The solution was stirred for 5 min, then Ac₂O (0.10 g, 1 mmol) was added and the stirring was continued at room temperature for 3 h. A saturated aqueous solution of NaHCO₃ (15 mL) was added and the mixture was extracted with EtOAc (3×20 mL). The combined organic phases were concentrated and the afforded crude was purified by flash column chromatography on silica gel which gave the title compound (0.042 g, 47%).

¹H NMR (DMSO-d₆, 400 MHz): δ 11.70 (1H, brs), 10.29 (1H, s), 8.68 (1H, d, J=2 Hz), 8.53 (1H, d, J=2 Hz), 4.69-4.62 (1H, m), 2.41-2.32 (2H, m), 2.07 (3H, s), 1.84-1.81 (2H, m), 1.65-1.62 (2H, m), 1.10-1.04 (3H, m), 0.86 (9H, s).

MS: m/z 359 [M+1]⁺.

The following compounds were prepared by acylation of the corresponding aniline derivative according to the procedure described in Example 33:

| | | | | MS |
|---|---|---|---|---|
| Ex | Structure | Name | Yield | [M + 1]⁺ |
| 34 | 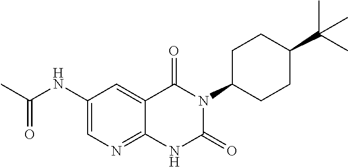 [SMILES: CC(=O)Nc1cnc2[nH]c(=O)n([C@H]3CC[C@@H](C(C)(C)C)CC3)c(=O)c2c1] | N-[3-(cis-4-tert-Butyl- cyclohexyl)-2,4-dioxo-1,2,3,4- tetrahydro-pyrido[2,3- d]pyrimidin-6-yl)-acetamide (34) | 44% | 359 |
| | ¹H NMR (DMSO-d₆, 400 MHz): δ 11.79 (1H, s), 10.29 (1H, s), 8.67 (1H, d, J = 2.4 Hz), | | | |
| | 8.55 (1H, d, J = 2.4 Hz), 4.89-4.86 (1H, m), 2.20-2.32 (2H, m), 2.08 (3H, s), 1.76-1.70 | | | |
| | (2H, m), 1.52-1.46 (4H, m), 1.33-1.30 (1H, m), 0.86 (9H, s). | | | |
| 35 | 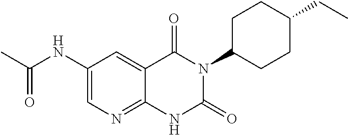 | N-[3-(trans-4-ethyl-cyclohexyl)- 2,4-dioxo-1,2,3,4-tetrahydro- pyrido[2,3-d]pyhmidin-6-yl]- acetamide (35) | 50% | 331 |
| | ¹H NMR (DMSO-d₆, 400 MHz): δ 11.79 (1H, s), 10.30 (1H, s), 8.68 (1H, d, J = 2 Hz), 8.54 | | | |
| | (1H, d, J = 2 Hz), 4.72-4.65 (1H, m), 2.41-2.37 (2H, m), 2.08 (3H, s), 1.83-1.80 (2H, m), | | | |
| | 1.58-1.55 (2H, m), 1.25-1.10 (3H, m), 1.0-0.9 (2H, m), 0.87 (3H, t, J = 7.2 Hz). | | | |
| 36 | 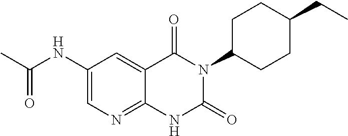 | N-(3-((1s,4s)-4- ethylcyclohexyl)-2,4-dioxo- 1,2,3,4-tetrahydropyrido[2,3- d]pyrimidin-6-yl)acetamide (36) | 53% | 331 |
| | ¹H NMR (DMSO-d₆, 400 MHz): δ 11.80 (1H, s), 10.29 (1H, s), 8.68 (1H s), 8.55 (1H, s), | | | |
| | 4.67-4.70 (1H, m), 2.51-2.49 (2H, m), 2.08 (3H, s), 1.71-1.67 (2H, m), 1.54-1.48 (5H m), | | | |
| | 1.37-1.30 (2H, m), 0.88 (3H, t, J = 6.8 Hz). | | | |
| 37 | 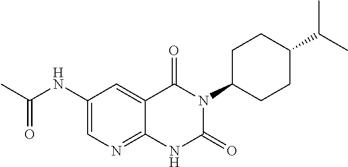 [SMILES: CC(=O)Nc1cnc2[nH]c(=O)n([C@H]3CC[C@H](C(C)C)CC3)c(=O)c2c1] | N-(3-((1R,4R)-4- isopropylcyclohexyl)-2,4-dioxo- 1,2,3,4-tetrahydropyrido[2,3- d]pyrimidin-6-yl)acetamide (37) | 50% | 345 |
| | ¹H NMR (DMSO-d₆, 400 MHz): δ 11.79 (1H, s), 10.29 (1H, s), 8.68 (1H, d, J = 2.4 Hz), | | | |
| | 8.54 (1H, d, J = 2.4 Hz), 4.75-4.60 (1H, m), 2.41-2.37 (2H, m), 2.07 (3H, s), 1.77 (2H, | | | |
| | m), 1.65-1.61 (2H, m), 1.44 (1H, m), 1.17-1.08 (3H, m), 0.86 (6H, d, J = 8 Hz). | | | |
| 38 | 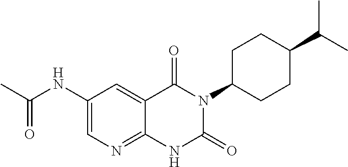 [SMILES: CC(=O)Nc1cnc2[nH]c(=O)n([C@H]3CC[C@@H](C(C)C)CC3)c(=O)c2c1] | N-(3-((1S,4S)-4- isopropylcyclohexyl)-2,4-dioxo- 1,2,3,4-tetrahydropyrido[2,3- d]pyrimidin-6-yl)acetamide (38) | 53% | 345 |
| | ¹H NMR (DMSO-d₆, 400 MHz): δ 11.80 (1H, s), 10.29 (1H, s), 8.66 (1H, d, J = 2.4 Hz), | | | |
| | 8.54 (1H, d, J = 2.4 Hz), 4.76-4.72 (1H, m), 2.49-2.40 (2H, m), 2.07 (3H, s), 1.98-1.86 | | | |
| | (3H, m), 1.42-1.30 (4H, m), 1.17-1.14 (1H, m), 0.89 (6H, d, J = 8 Hz). | | | |
| 39 | 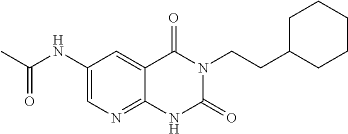 [SMILES: CC(=O)Nc1cnc2[nH]c(=O)n(CCC3CCCCC3)c(=O)c2c1] | N-(3-(2-cyclohexylethyl)-2,4- dioxo-1,2,3,4- tetrahydropyrido[2,3- d]pyrimidin-6-yl)acetamide (39) | 10% | 331 |

¹H NMR (DMSO-d₆, 400 MHz): δ 11.88 (1H, s), 10.30 (1H, s), 8.69 (1H, d, J = 2.4 Hz),
8.57 (1H, d, J = 2.4 Hz), 3.90-3.87 (2H, m), 2.08 (3H, s), 1.76-1.64 (5H, m), 1.47-1.44
(2H, m), 1.22-1.11 (4H, m), 0.95-0.87 (2H, m).

### Example 40

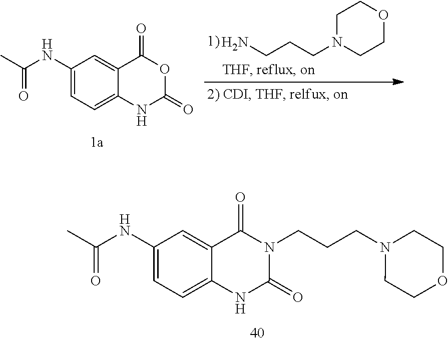 [SMILES: CC(=O)Nc1ccc2[nH]c(=O)oc(=O)c2c1 ; NCCCN1CCOCC1 ; CC(=O)Nc1ccc2[nH]c(=O)n(CCCN3CCOCC3)c(=O)c2c1]

### N-(3-(3-Morpholinopropyl)-2,4-dioxo-1,2,3,4-tetrahydroquinazolin-6-yl)acetamide (40)

A solution of compound 1a (150 mg, 0.682 mmol) and 3-(4-morpholinyl)propylamine (97.4 mg, 0.675 mmol, 0.99 eq) in THF (5 mL) was heated under reflux overnight, then cooled to room temperature. Carbonyldiimidazole (162 mg, 1.02 mmol, 1.5 eq) was added and the mixture was heated under reflux. After 24 hours, the reaction mixture was cooled to rt and the resulting precipitate was collected by filtration, washed with water, CH₂Cl₂ and Et₂O and dried in vacuo. The resultant residue was diluted with water and extracted with EtOAc. The organic layer was dried (MgSO₄) and concentrated. Purification of the afforded crude by flash chromatography gave the title compound (75 mg, 0.22 mmol, 32%).

¹H NMR (500 MHz, DMSO-d6) δ (ppm) 11.34 (s, 1H), 10.10 (s, 1H), 8.24 (d, J=2.4 Hz, 1H), 7.79 (dd, J=8.8, 2.4 Hz, 1H), 7.13 (d, J=8.8 Hz, 1H), 3.95 (t, J=7.2 Hz, 2H), 3.50-3.44 (m, 4H), 2.35-2.30 (m, 6H), 2.05 (s, 3H), 1.74 (quintet, J=7.0 Hz, 2H); LC-MS m/z: 347 [M+H]⁺.

The following compounds were prepared from compound 1a and the indicated amine R¹—NH₂ according to the procedure described in Example 40:

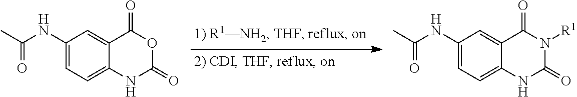

| | | | | MS |
|---|---|---|---|---|
| Ex. | Structure/Name | R¹—NH₂ | Yield | [M + 1]⁺ |
| 41 | 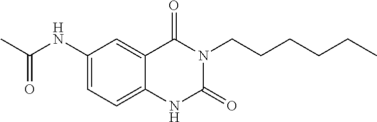 [SMILES: CCCCCCn1c(=O)[nH]c2ccc(NC(C)=O)cc2c1=O] | 1-Hexylamine | 31% | 304 |
| | N-(3-hexyl-2,4-dioxo-1,2,3,4-tetrahydroquinazolin-6- | | | |
| | yl)acetamide (41) | | | |
| | ¹H NMR (500 MHz, DMSO-d6) δ (ppm) 11.35 (s, 1H), 10.10 (s, 1H), 8.23 (d, J = 2.4 Hz, | | | |
| | 1H), 7.79 (dd, J = 8.8, 2.4 Hz, 1H), 7.12 (d, J = 8.8 Hz, 1H), 3.87 (t, J = 7.5 Hz, 2H), | | | |
| | 2.05 (s, 3H), 1.56 (m, 2H), 1.28 (m, 6H), 0.87 (t, J = 7.8 Hz, 3H). | | | |
| 42 | 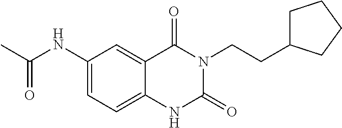 [SMILES: CC(=O)Nc1ccc2[nH]c(=O)n(CCC3CCCC3)c(=O)c2c1] | 2-Cyclopentyl- ethanamine | 28% | 316 |
| | N-(3-(2-cyclopentylethyl)-2,4-dioxo-1,2,3,4- | | | |
| | tetrahydroquinazolin-6-yl)acetamide (42) | | | |
| | ¹H NMR (500 MHz, DMSO-d6) δ (ppm) 11.35 (s, 1H), 10.10 (s, 1H), 8.22 (d, J = 2.0 Hz, | | | |
| | 1H), 7.79 (dd, J = 8.8, 2.0 Hz, 1H), 7.12 (d, J = 8.8 Hz, 1H), 3.89 (t, J = 7.5 Hz, 2H), | | | |
| | 2.05 (s, 3H), 1.78 (m, 3H), 1.58 (m, 4H), 1.48 (m, 2H), 1.12 (m, 2H); | | | |
| 43 | 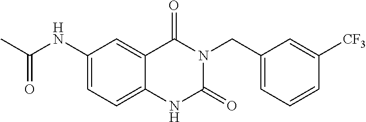 [SMILES: CC(=O)Nc1ccc2[nH]c(=O)n(Cc3cccc(C(F)(F)F)c3)c(=O)c2c1] | 3- Trifluoromethyl- benzylamine | 51% | 378 |
| | N-(2,4-dioxo-3-(3-(trifluoromethyl)benzyl)-1,2,3,4- | | | |
| | tetrahydroquinazolin-6-yl)acetamide (43) | | | |
| | ¹H NMR (500 MHz, DMSO-d6) δ (ppm) 11.46 (s, 1H), 10.13 (s, 1H), 8.27 (d, J = 1.4 Hz, | | | |
| | 1H), 7.81 (d, J = 8.7 Hz, 1H), 7.70 (s, 1H), 7.64-7.54 (m, 4H), 7.17 (d, J = 8.8 Hz, 1H), | | | |
| | 5.17 (s, 2H), 2.05 (s, 3H) | | | |
| 44 | 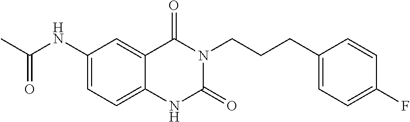 [SMILES: CC(=O)Nc1ccc2[nH]c(=O)n(CCCc3ccc(F)cc3)c(=O)c2c1] | 3-(4- Fluorophenyl)- propan-1- amine | 75% | 356 |
| | N-(3-(3-(4-fluorophenyl)propyl)-2,4-dioxo-1,2,3,4- | | | |
| | tetrahydroquinazolin-6-yl)acetamide (44) | | | |
| | ¹H NMR (500 MHz, DMSO-d6) δ (ppm) 11.30 (s, 1H), 10.09 (s, 1H), 8.23 (d, J = 1.9 Hz, | | | |
| | 1H), 7.79 (dd, J = 8.8, 1.9 Hz, 1H), 7.27 (m, 2H), 7.10 (m, 3H), 3.92 (t, J = 7.2 Hz, 2H), | | | |
| | 2.63 (t, J = 7.5 Hz, 2H), 2.05 (s, 3H), 1.87 (quintet, J = 7.2 Hz, 2H) | | | |
| 45 | 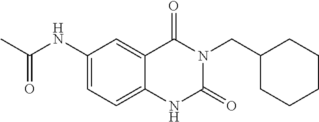 [SMILES: CC(=O)Nc1ccc2[nH]c(=O)n(CC3CCCCC3)c(=O)c2c1] | Cyclohexyl- methanamine | 78% | 316 |
| | N-(3-(cyclohexylmethyl)-2,4-dioxo-1,2,3,4- | | | |
| | tetrahydroquinazolin-6-yl)acetamide (45) | | | |
| | ¹H NMR (500 MHz, DMSO-d6) δ (ppm) 11.33 (s, 1H), 10.08 (s, 1H), 8.22 (d, J = 2.4 Hz, | | | |
| | 1H), 7.79 (dd, J = 8.8, 2.4 Hz, 1H), 7.12 (d, J = 8.8 Hz, 1H), 3.77 (d, J = 7.3 Hz, 2H), 2.05 | | | |
| | (s, 3H), 1.76-1.73 (m, 1H), 1.67-1.65 (m, 2H), 1.59-1.56 (m, 3H), 1.19-1.10 (m, 3H), | | | |
| | 1.02-0.96 (m, 2H); ¹³C-NMR (125 MHz, DMSO-d6) δ (ppm) 168.2, 162.0, 150.1, 134.9, | | | |
| | 134.2, 126.5, 116.6, 115.4, 113.6, 45.5, 35.8, 30.2, 25.8, 25.2, 23.8 | | | |
| 46 | 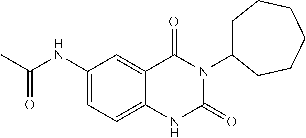 [SMILES: CC(=O)Nc1ccc2[nH]c(=O)n(C3CCCCCC3)c(=O)c2c1] | Cycloheptan- amine | 64% | 648 |
| | N-(3-Cycloheptyl-2,4-dioxo-1,2,3,4-tetrahydro- | | | |
| | quinazolin-6-yl)acetamide (46) | | | |
| | ¹H NMR (500 MHz, DMSO-d6) δ (ppm) 11.25 (s, 1H), 10.08 (s, 1H), 8.20(d, J = 2.4 Hz, | | | |
| | 1H), 7.78 (dd, J = 8.8, 2.3 Hz, 1H), 7.09 (d, J = 8.8, 1H), 4.89 (bs, 1H), 2.35-2.27 (m, 2H), | | | |
| | 2.04 (s, 3H), 1.76-1.66 (m, 4H), 1.61-1.54 (m, 4H), 1.52-1.41 (m, 2H); ¹³C-NMR (125 | | | |
| | MHz, DMSO-d6) δ (ppm) 168.2, 161.7, 149.8, 135.0, 134.1, 126.4, 116.7, 115.1, 54.0, | | | |
| | 31.3, 27.5, 25.9, 23.8 | | | |
| 47 | 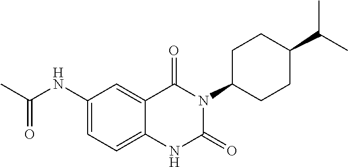 [SMILES: CC(=O)Nc1ccc2[nH]c(=O)n([C@H]3CC[C@@H](C(C)C)CC3)c(=O)c2c1] | (1s,4s)-4- Isopropyl- cyclohexan- amine | 27% | 344 |
| | N-(3-((1s,4s)-4-Isopropylcyclohexyl)-2,4-dioxo-1,2,3,4- | | | |
| | tetrahydroquinazolin-6-yl)acetamide (47) | | | |
| | ¹H NMR (500 MHz, DMSO-d6) δ (ppm) 10.06 (s, 1H), 8.21 (d, J = 2.4 Hz, 1H), 7.76 (dd, | | | |
| | J = 8.8, 2.4 Hz, 1H), 7.10 (d, J = 8.8 Hz, 1H), 4.77 (m, 1H), 2.53-2.46 (m, 2H), 2.04 (s, | | | |
| | 3H), 1.97 (m, 1H), 1.90 (d, J = 13.6 Hz, 2H), 1.44-1.34 (m, 4H), 1.17 (m, 1H), 0.92 (d, J = | | | |
| | 6.5 Hz, 6H) | | | |
| 48 | 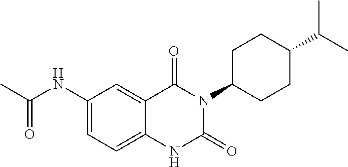 [SMILES: CC(=O)Nc1ccc2[nH]c(=O)n([C@H]3CC[C@H](C(C)C)CC3)c(=O)c2c1] | (1r,4r)-4- isopropyl- cyclohexan- amine | 40% | 344 |
| | N-(3-((1r,4r)-4-Isopropylcyclohexyl)-2,4-dioxo-1,2,3,4- | | | |
| | tetrahydroquinazolin-6-yl)acelamide (48) | | | |
| | ¹H NMR (500 MHz, DMSO-d6) δ (ppm) 11.26 (s, 1H), 10.09 (s, 1H), 8.19 (d, J = 2.4 Hz, | | | |
| | 1H), 7.79 (dd, J = 8.8, 2.4 Hz, 1H), 7.08 (d, J = 8.8 Hz, 1H), 4.71 (m, 1H), 2.42 (m, 2H), | | | |
| | 2.04 (s, 3H), 1.77 (m, 2H), 1.61 (d, J = 11.4 Hz, 2H), 1.46 (m, 1H), 1.08 (m, 4H), 0.89 | | | |
| | (d, J = 6.8 Hz, 6H). | | | |
| 49 | 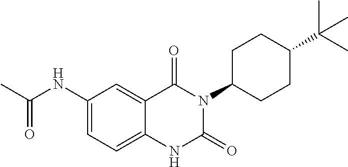 [SMILES: CC(=O)Nc1ccc2[nH]c(=O)n([C@H]3CC[C@H](C(C)(C)C)CC3)c(=O)c2c1] | (1r,4r)-4-(tert- butyl)- cyclohexan- amine | 33% | 356 |
| | N-(3-((1r,4r)-4-(tert-butyl)cyclohexyl)-2,4-dioxo-1,2,3,4- | | | |
| | tetrahydroquinazolin-6-yl)acetamide (49) | | | |
| | ¹H NMR (500 MHz, DMSO-d6) δ (ppm) 11.23 (s, 1H), 10.06 (s, 1H), 8.18 (d, J = 2.4 Hz, | | | |
| | 1H), 7.79 (dd, J = 8.8, 2.4 Hz, 1H), 7.08 (d, J = 8.8 Hz, 1H), 4.71 (m, 1H), 2.42 (m, 2H), | | | |
| | 2.04 (s, 3H), 1.84 (d, J = 10.6 Hz, 1H), 1.64 (d, J = 9.9 Hz, 2H), 1.14-1.04 (m, 3H), 0.88 | | | |
| | (s, 9H). | | | |
| 50 | 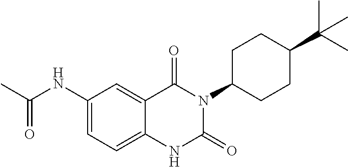 [SMILES: CC(=O)Nc1ccc2[nH]c(=O)n([C@H]3CC[C@@H](C(C)(C)C)CC3)c(=O)c2c1] | (1s,4s)-4-(tert- butyl)- cyclohexan- amine | 15% | 356 |
| | N-(3-((1s,4s)-4-(Tert-butyl)cyclohexyl)-2,4-dioxo- | | | |
| | 1,2,3,4-tetrahydroquinazolin-6-yl)acetamide (50) | | | |
| | ¹H NMR (500 MHz, DMSO-d6) δ (ppm) 11.21 (s, 1H), 10.06 (s, 1H), 8.21 (d, J = 2.4 Hz, | | | |
| | 1H), 7.77 (dd, J = 8.8, 2.4 Hz, 1H), 7.09 (d, J = 8.8 Hz, 1H), 4.92 (m, 1H), 2.33 (m, 2H), | | | |
| | 2.05 (s, 3H), 1.73 (m, 2H), 1.50 (m, 4H), 1.33 (m, 1H), 0.92 (s, 9H) | | | |
| 51 | 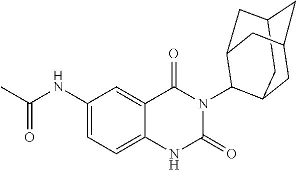 [SMILES: CC(=O)Nc1ccc2[nH]c(=O)n(C3C4CC5CC(C4)CC3C5)c(=O)c2c1] | 2-Adamantyl- amine | 26% | 354 |
| | N-(3-((1r,3r,5r,7r)-adamantan-2-yl)-2,4-dioxo-1,2,3,4- | | | |
| | tetrahydroquinazolin-6-yl)acetamide (51) | | | |
| | ¹H NMR (500 MHz, DMSO-d6) δ (ppm) 11.13 (s, 1H), 10.05 (s, 1H), 8.20 (d, J = 2.4 Hz, | | | |
| | 1H), 7.74 (dd, J = 8.8, 2.4 Hz, 1H), 7.07 (d, J = 8.7 Hz, 1H), 4.73 (s, 1H), 2.42 (s, 2H), | | | |
| | 2.30 (d, J = 12.5 Hz, 2H), 2.05 (s, 3H), 1.92-1.85 (m, 7H) 1.74-1.70 (m, 2H), 1.59-1.52 | | | |
| | (m, 2H). | | | |
| 52 | 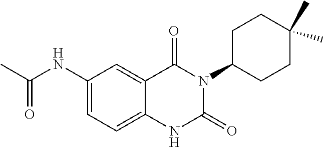 | 4,4-Dimethyl- cyclohexan- amine | 10% | 328 |
| | N-(3-(4,4-dimethylcyclohexyl)-2,4-dioxo-1,2,3,4- | | | |
| | tetrahydroquinazolin-6-yl)acetamide (52) | | | |
| | ¹H NMR (500 MHz, DMSO-d6) δ (ppm) 10.09 (s, 1H), 8.21 (d, J = 2.4 Hz, 1H), 7.77 (dd, | | | |
| | J = 8.8, 2.4 Hz, 1H), 7.09 (d, J = 8.8 Hz, 1H), 4.69 (m, 1H), 2.60 (qd, J = 13.0, 3.2 Hz, | | | |
| | 2H), 2.05 (s, 3H), 1.44 (d, J = 12.4 Hz, 2H), 1.38 (m, 2H), 1.28 (td, J = 13.3, 3.6 Hz, | | | |
| | 2H), 1.01 (s, 3H), 0.94 (s, 9H) | | | |
| 53 | 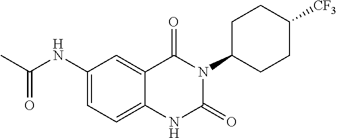 [SMILES: CC(=O)Nc1ccc2[nH]c(=O)n([C@H]3CC[C@H](C(F)(F)F)CC3)c(=O)c2c1] | (1r,4r)-4- (trifluoro- methyl)- cyclohexan- amine | 22% | 368 |
| | N-(2,4-dioxo-3-((1r,4r)-4-(trifluoromethyl)cyclohexyl)- | | | |
| | 1,2,3,4-tetrahydroquinazolin-6-yl)acetamide (53) | | | |
| | ¹H NMR (500 MHz, DMSO-d6) δ (ppm) 11.30 (s, 1H), 10.10 (s, 1H), 8.20 (d, J = 2.4 Hz, | | | |
| | 1H), 7.80 (dd, J = 8.8, 2.4 Hz, 1H), 7.09 (d, J = 8.8 Hz, 1H), 4.76 (m, 1H), 2.49 (m, 2H), | | | |
| | 2.30 (m, 1H), 2.04 (s, 3H), 1.98 (d, J = 11.8 Hz, 2H), 1.71 (d, J = 10.2 Hz, 2H), 1.39 (qd, | | | |
| | J = 12.8, 3.4 Hz, 2H) | | | |
| 54 | 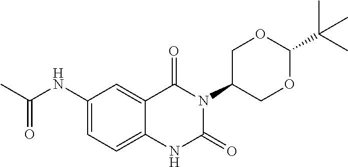 [SMILES: CC(=O)Nc1ccc2[nH]c(=O)n([C@H]3CO[C@H](C(C)(C)C)OC3)c(=O)c2c1] | (2r,5r)-2-(Tert- butyl)-1,3- dioxan-5- amine | 57% | 276 |
| | N-(3-((2r,5r)-2-(tert-butyl)-1,3-dioxan-5-yl)-2,4-dioxo- | | | |
| | 1,2,3,4-tetrahydroquinazolin-6-yl)acetamide (54) | | | |
| | ¹H NMR (500 MHz, DMSO-d6) δ (ppm) 11.39 (s, 1H), 10.11 (s, 1H), 8.22 (d, J = 2.4 Hz, | | | |
| | 1H), 7.79 (dd, J = 8.8, 2.4 Hz, 1H), 7.10 (d, J = 8.8 Hz, 1H), 5.01 (m, 1H), 4.52 (dd | | | |
| | appearing as t, J = 10.7 Hz, 2H), 4.22 (s, 1H), 4.12 (dd appearing as q, J = 5.3 Hz, 2H), | | | |
| | 2.05 (s, 3H), 0.91 (s, 9H); | | | |
| 55 | 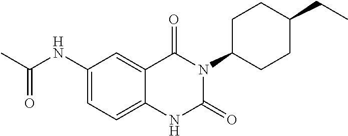 [SMILES: CC[C@H]1CC[C@@H](n2c(=O)[nH]c3ccc(NC(C)=O)cc3c2=O)CC1] | (1s,4s)-4- Ethylcyclohex- anamine | 18% | 330 |
| | N-(3-((1s,4s)-4-Ethylcyclohexyl)-2,4-dioxo-1,2,3,4- | | | |
| | tetrahydroquinazolin-6-yl)acetamide (55) | | | |
| | ¹H NMR (500 MHz, DMSO-d6) δ (ppm) 11.24 (s, 1H), 10.09 (s, 1H), 8.21 (d, J = 2.4 Hz, | | | |
| | 1H), 7.77 (dd, J = 8.8, 2.4 Hz, 1H), 7.09 (d, J = 8.8 Hz, 1H), 4.72 (m, 1H), 2.53 (m, 2H), | | | |
| | 2.05 (s, 3H), 1.70 (d, J = 12.6 Hz), 1.50 (m, 5H), 1.33 (d, J = 9.7 Hz, 2H), 0.89 (t, J = 7.3 | | | |
| | Hz, 3H). | | | |
| 56 | 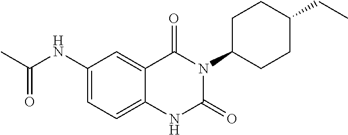 [SMILES: CC[C@H]1CC[C@H](n2c(=O)[nH]c3ccc(NC(C)=O)cc3c2=O)CC1] | (1r,4r)-4- Ethylcyclohex- anamine | 16% | 330 |
| | N-(3-((1r,4r)-4-Ethylcyclohexyl)-2,4-dioxo-1,2,3,4- | | | |
| | tetrahydroquinazolin-6-yl)acetamide (56) | | | |
| | ¹H NMR (500 MHz, DMSO-d6) δ (ppm) 11.26 (s, 1H), 10.09 (s, 1H), 8.19 (d, J = 2.4 Hz, | | | |
| | 1H), 7.78 (dd, J = 8.8, 2.4 Hz, 1H), 7.08 (d, J = 8.8 Hz, 1H), 4.73 (m, 1H), 2.42 (qd, J = | | | |
| | 12.6 and 3.2 Hz, 2H), 2.04 (s, 3H), 1.83 (d, J = 12.0 Hz, 2H), 1.58 (d, J = 9.6 Hz, 2H) | | | |
| | 1.23 (m, 2H), 1.16 (m, 1H), 0.98 (qd, J = 12.8, 3.1 Hz, 2H), 0.89 (t, J = 7.4 Hz, 3H). | | | |
| 57 | 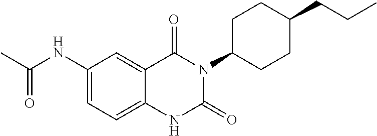 [SMILES: CCC[C@H]1CC[C@@H](n2c(=O)[nH]c3ccc(NC(C)=O)cc3c2=O)CC1] | (1r,4s)-4- Propyl- cyclohexan- amine | 15% | 344 |
| | N-(2,4-Dioxo-3-((1r,4s)-4-propylcyclohexyl)-1,2,3,4- | | | |
| | tetrahydroquinazolin-6-yl)acetamide (57) | | | |
| | ¹H NMR (500 MHz, DMSO-d6) δ (ppm) 11.23 (s, 1H), 10.09 (s, 1H), 8.22 (d, J = 2.4 Hz, | | | |
| | 1H), 7.76 (dd, J = 8.8, 2.4 Hz, 1H), 7.10 (d, J = 8.8 Hz, 1H), 4.73 (m, 1H), 2.53 (m, 2H), | | | |
| | 2.05 (s, 3H), 1.67 (m, 3H), 1.55-1.42 (m, 4H), 1.30 (m, 4H), 0.93 (t, J = 7.3 Hz, 3H). | | | |
| 58 | 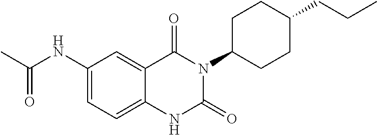 [SMILES: CCC[C@H]1CC[C@H](n2c(=O)[nH]c3ccc(NC(C)=O)cc3c2=O)CC1] | (1s,4r)-4- Propyl- cyclohexan- amine | 19% | 344 |
| | N-(2,4-Dioxo-3-((1s,4r)-4-propylcydohexyl)-1,2,3,4- | | | |
| | tetrahydroquinazolin-6-yl)acetamide (58) | | | |
| | ¹H NMR (500 MHz, DMSO-d6) δ (ppm) 11.26 (s, 1H), 10.09 (s, 1H), 8.19 (d, J = 2.4 Hz, | | | |
| | 1H), 7.78 (dd, J = 8.8, 2.4 Hz, 1H), 7.08 (d, J = 8.8 Hz, 1H), 4.72 (m, 1H), 2.42 (qd, J = | | | |
| | 8.5, 3.0 Hz, 2H), 2.04 (s, 3H), 1.81 (d, J = 12.0 Hz, 2H), 1.58 (d, J = 9.8 Hz), 1.36-1.16 | | | |
| | (m, 5H), 0.99 (qd, J = 12.5, 3.0 Hz, 2H), 0.88 (t, J = 7.3 Hz, 3H); | | | |
| 59 | 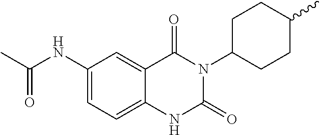 [SMILES: CC(=O)Nc1ccc2[nH]c(=O)n(C3CCC(C)CC3)c(=O)c2c1] | 4-Methyl- cyclohexan- amine | 88% | 316 |
| | N-(3-(4-methylcyclohexyl)-2,4-dioxo-1,2,3,4- | | | |
| | tetrahydroquinazolin-6-yl)acetamide (59) | | | |
| | ¹H NMR (500 MHz, DMSO-d6) δ (ppm) 11.23 (s, 1H), 10.07 (s, 1H), 8.26-8.24 (m, 1H), | | | |
| | 8.20 (dd, J = 11.7, 2.4 Hz, 1H), 7.80-7.76 (m, 1H), 7.71-7.68 (m, 1H), 7.10 (dd, J = 8.8, | | | |
| | 5.2 Hz, 1H), 7.04-7.01 (m, 1H), 4.74-4.69 (m, 1H), 2.66-2.58 (m, 1H), 2.46 (dd, J = 12.6, | | | |
| | 3.1 Hz, 1H), 2.05 (s, 3H), 1.95-1.87 (m, 1H), 1.77-1.66 (m, 2H), 1.63-1.48 (m, 4H), 1.45- | | | |
| | 1.33 (m, 3H), 1.08-1.00 (m, 3H), 0.98-0.91 (d, J = 6.9 Hz, 1H), 0.90-0.87 (m, 2H); | | | |
| 60 | 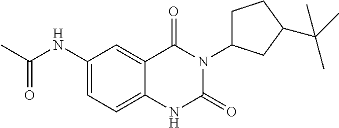 [SMILES: CC(=O)Nc1ccc2[nH]c(=O)n(C3CCC(C(C)(C)C)C3)c(=O)c2c1] | 3-(Tert- butyl)cyclo- pentanamine | 54% | 342 |
| | N-(3-(3-(Tert-butyl)cyclopentyl)-2,4-dioxo-1,2,3,4- | | | |
| | tetrahydroquinazolin-6-yl)acetamide (60) | | | |
| | ¹H NMR (500 MHz, DMSO-d6) δ (ppm) 11.26 (s, 1H), 10.09 and 10.08 (2xs, 1H), 8.23 | | | |
| | (m, 1H), 7.76 (m, 1H), 7.10 (m, 1H), 5.25 (m, 1H), 2.36-1.23 (m, 7H), 2.05 (s, 3H), 0.89 | | | |
| | and 0.86 (2xs, 9H). | | | |
| 61 | 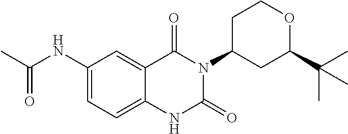 [SMILES: CC(=O)Nc1ccc2[nH]c(=O)n([C@H]3CCO[C@@H](C(C)(C)C)C3)c(=O)c2c1] | (2r,4s)-2-(Tert- butyl)tetra- hydro-2H- pyran-4-amine | 53% | 360 |
| | N-(3-((2R,4S)-2-(Tert-butyl)tetrahydro-2H-pyran-4-yl)- | | | |
| | 2,4-dioxo-1,2,3,4-tetrahydroquinazolin-6-yl)acetamide | | | |
| | (61) | | | |
| | 1H NMR (500 MHz, DMSO-d6) δ(ppm) 11.30 (s, 1H), 10.10 (s, 1H), 8.21 (d, J =2.4 Hz, | | | |
| | 1H), 7.79 (dd, J =8.8, 2.4 Hz, 1H), 7.10 (d, J =8.8 Hz, 1H), 5.00 (m, 1H), 4.04 (dd, J = | | | |
| | 11.3, 3.8 Hz, 1H), 3.40 (m, 1H), 2.96 (dd, J =11.2, 1.4 Hz, 1H), 2.59 (dq, J =4.8, 12.2 | | | |
| | Hz, 1H), 2.36 (q, J =12.2 Hz, 1H), 2.04 (s, 3H), 1.57 (m, 1H), 1.48 (m, 1H), 0.88 (s, | | | |
| | 9H). | | | |
| 62 | 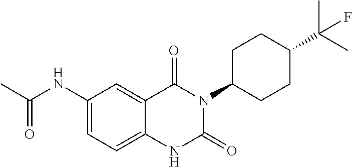 [SMILES: CC(=O)Nc1ccc2[nH]c(=O)n([C@H]3CC[C@H](C(C)(C)F)CC3)c(=O)c2c1] | (1r,4r)-4-(2- Fluoropropan- 2-yl)- cyclohexan- amine | 54% | 362 |
| | N-(3-((1r,4r)-4-(2-Fluoropropan-2-yl)cyclohexyl)-2,4- | | | |
| | dioxo-1,2,3,4-tetrahydroquinazolin-6-yl)acetamide (62) | | | |

¹H NMR (500 MHz, DMSO-d6) δ (ppm) 11.24 (s, 1H), 10.06 (s, 1H), 8.19 (d, J = 2.4 Hz,
1H), 7.79 (dd, J = 8.8, 2.4 Hz, 1H), 7.09 (d, J = 8.8 Hz, 1H), 4.73 (m, 1H), 2.45 (qd, J =
2.9, 12.4 Hz, 2H), 2.05 (s, 3H), 1.86 (d, J = 12.1 Hz, 2H) 1.66 (d, J = 10.1 Hz, 2H), 1.54
(q, J = 12.0 Hz, 1H), 1.30 (d, J = 22.2 Hz, 6H), 1.19 (qd, J = 2.9, 12.6 Hz, 2H).

### Example 63

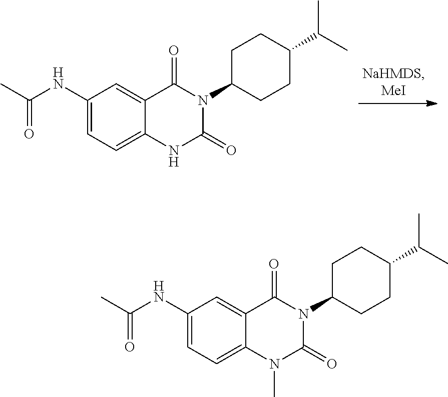

### N-(3-((1 r,4r)-4-isopropylcyclohexyl)-1-methyl-2,4-dioxo-1,2,3,4-tetrahydroquinazolin-6-yl)acetamide (63)

A 1M solution of NaHMDS in THF (0.11 mL, 0.11 mmol) was added dropwise at room temperature to a solution of N-(3-((1r,4r)-4-isopropylcyclohexyl)-2,4-dioxo-1,2,3,4-tetrahydroquinazolin-6-yl)acetamide (34 mg, 99 μmol) in DMF (1 mL). The solution was stirred at room temperature for 15 minutes, then iodomethane (8 μL, 0.13 mmol) was added and the stirring was continued for three days. The reaction mixture was diluted with water (10 mL) and the resulting precipitate was collected by filtration and washed with water and Et₂O. Purification by flash chromatography (0-10% MeOH/CH₂Cl₂) gave the title compound (8.1 mg, 23%).

¹H NMR (500 MHz, DMSO-d6) δ (ppm) 10.14 (s, 1H), 8.28 (d, J=2.6 Hz, 1H), 7.93 (dd, J=9.0, 2.6 Hz, 1H), 7.38 (d, J=9.0 Hz, 1H), 4.77 (m, 1H), 3.51 (s, 3H), 2.43 (m, 1H), 2.06 (s, 3H), 1.78 (m, 2H), 1.63 (m, 2H), 1.47 (m, 1H), 1.25 (m, 1H), 1.10 (m, 3H), 0.88 (d, J=6.8 Hz, 6H); LC-MS m/z: 358 [M+H]+.

### Biology Example 1

Activity against T. cruzi. Rat skeletal myoblasts (L-6 cells) were seeded in 96-well microtitre plates at 2000 cells/well in 100 μL RPMI 1640 medium with 10% FBS and 2 mM 1-glutamine. After 24 h the medium was removed and replaced by 100 μl per well containing 5000 trypomastigote forms of T. cruzi Tulahuen strain C2C4 containing the 3-galactosidase (Lac Z) gene (Buckner et al. 1996). After 48 h the medium was removed from the wells and replaced by 100 μl fresh medium with or without a serial drug dilution of eleven 3-fold dilution steps covering a range from 100 to 0.002 μg/ml. After 96 h of incubation the plates were inspected under an inverted microscope to assure growth of the controls and sterility. Then the substrate CPRG/Nonidet (50 μl) was added to all wells. A color reaction developed within 2-6 h and could be read photometrically at 540 nm. Data were analyzed with the graphic programme Softmax Pro (Molecular Devices), which calculated IC₅₀ values by linear regression (Huber 1993) from the sigmoidal dose inhibition curves. Benznidazole is used as control (IC₅₀ 0.5±0.2 ug/ml).
Buckner, F. S., C. L. Verlinde, A. C. La Flamme, and W. C. Van Voorhis. 1996. Efficient technique for screening drugs for activity against Trypanosoma cruzi using parasites expressing beta-galactosidase, p. 2592-2597, vol. 40.
Huber, W. Koella, J. C. 1993. A comparison of the three methods of estimating EC50 in studies of drug resistance of malaria parasites. Acta Trop. 55, 257-261.

Table 1 shows, as examples, IC₅₀ data of some of the compounds of the present invention. The compounds have an excellent trypanosomicidal activity in vitro.

TABLE 1

| | Activity | | Activity |
|---|---|---|---|
| Example | ug/ml | Example | ug/ml |
| 1c | 0.692 | 2 | 0.724 |
| 3 | 0.162 | 4 | 2.81 |
| 5 | 0.163 | 6 | 9.62 |
| 7 | 63.6 | 8 | ND |
| 9 | 0.339 | 10 | 0.293 |
| 11 | 6.44 | 12a | ND |
| 12b | 7.7 | 13c | 5.23 |
| 14c | 5.88 | 15b | 2.64 |
| 16c | 11.5 | 17 | 0.655 |
| 18 | 4.93 | 19 | 0.605 |
| 10 | 0.662 | 21c | 0.892 |
| 22 | 2.63 | 23b (rac) | 0.028 |
| 23b (cis) | 0.007 | 23b (trans) | 0.030 |
| 24 (rac) | 0.022 | 24 (cis) | 0.096 |
| 24 (trans) | 0.035 | 25 (rac) | 0.028 |
| 25 (trans) | 0.008 | 26e | 0.209 |
| 27 | 0.289 | 30 | ND |
| 31 | ND | 32 | ND |
| 33 | 0.196 | 34 | 0.36 |
| 35 | 0.1 | 36 | 0.110 |
| 37 | ND | 38 | ND |
| 39 | 0.755 | 40 | 55.3 |
| 41 | 0.881 | 42 | 0.713 |
| 43 | 0.804 | 44 | 0.943 |
| 45 | ND | 46 | 0.661 |
| 47 | 0.087 | 48 | 0.018 |
| 49 | 0.008 | 50 | 0.005 |
| 51 | 0.178 | 52 | 0.058 |
| 53 | 0.13 | 54 | ND |
| 55 | 0.3 | 56 | 0.015 |
| 57 | 0.06 | 58 | 0.007 |
| 59 | 0.329 | 60 | 0.007 |
| 61 | 0.744 | 62 | 0.933 |

1. A compound of the formula: 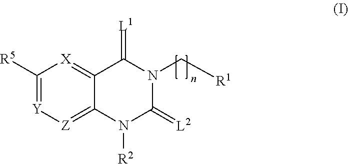
wherein
L¹ and L² are independently selected from O and S;
R¹ is C₃-C₆ straight or branched alkyl, C₃-C₇cycloalkyl, C₅-C₇cycloalkenyl, adamantly, phenyl or saturated heterocyclyl, any of which being optionally substituted with 1-3 substituents selected from halo, C₁-C₆ alkyl, C₃-C₆cycloalkyl, C₁-C₆haloalkyl, C₁-C₆alkoxy, ORy, SRy, N₃, NRxRy, CORy, COORy, and CONRxRy;
R² is H, methyl or ethyl;
R⁵ is NRxCORy, NRxRy, CH₂COCH₃, CH₂C≡N, or a 5- or 6-membered heteroaryl group which is optionally substituted with 1-3 substituents independently selected from halo, C₁-C₆ alkyl, C₃-C₆cycloalkyl, C₁-C₆haloalkyl, C₁-C₆alkoxy, ORy, SRy, N₃, NRxRy, CORy, COORy, and CONRxRy;
X, Y and Z are independently N or CH;
Rx is independently H or C₁-C₄alkyl;
Ry is independently H, C₁-C₄alkyl, phenyl or benzyl, either of which is optionally substituted with 1-3 substituents selected from halo, C₁-C₆alkyl, C₃-C₆cycloalkyl, C₁-C₆haloalkyl, C₁-C₆alkoxy, COC₁-C₆alkyl;
n is 0-3;
or a pharmaceutically acceptable salt, hydrate or N-oxide thereof.

2. The compound according to claim 1, wherein X, Y and Z are each CH.

3. The compound according to claim 1, wherein n is 2 and R¹ is cyclohexyl or cyclohexenyl any of which is optionally substituted with 1-3 substituents selected from halo, C₁-C₆ alkyl, C₃-C₆cycloalkyl, C₁-C₆haloalkyl, C₁-C₆alkoxy, ORy, SRy, N₃, NRxRy, CORy, COORy, and CONRxRy, wherein R_{y} and Rₓ are defined as in claim 1.

4. The compound according to claim 1, wherein n is 0.

5. The compound according to claim 4, wherein R¹ is cyclopentyl or cyclohexyl, any of which is optionally substituted, as defined

6. The compound according to claim 5, wherein R¹ is cyclohexyl which is substituted in the 4-position.

7. The compound according to claim 6, wherein R¹ is cyclohexyl which is substituted in the 4-position with C₁-C₄alkyl.

8. The compound according to claim 6, wherein R¹ is cyclohexyl which is substituted in the 4-position with isopropyl.

9. The compound according to claim 1, wherein R² is H.

10. The compound according to claim 1, wherein R⁵ is NH₂ or NHCOCH₃.

11. The compound according to any preceding claim, wherein L¹ and L² are O.

12. A pharmaceutical composition comprising the compound of claim 1 and a pharmaceutically acceptable vehicle or diluent therefor.

13. (canceled)

14. (canceled)

15. A method for the treatment or prophylaxis of Chagas disease comprising administering the compound of claim 1 to a subject suffering from, or likely to be exposed to, Chagas disease.
